(19) United States
(12) Patent Application Publication
(10) Pub. No.: US 2025/0171843 A1
(43) Pub. Date: May 29, 2025

(54) SINGLE-CELL NANOPARTICLE TARGETING-SEQUENCING (SENT-SEQ)

(71) Applicant: GEORGIA TECH RESEARCH CORPORATION, Atlanta, GA (US)

(72) Inventors: James Dahlman, Atlanta, GA (US); Curtis Dobrowolski, Boston, MA (US); Kalina Paunovska, Atlanta, GA (US)

(21) Appl. No.: 18/841,103

(22) PCT Filed: Feb. 24, 2023
(86) PCT No.: PCT/US2023/063221

§ 371 Date: Aug. 23, 2024

Related U.S. Application Data
(60) Provisional application No. 63/314,166, filed on Feb. 25, 2022.

(51) Int. Cl. C12Q 1/6869; G01N 33/543

(52) U.S. Cl. CPC C12Q 1/6869; G01N 33/54326

(57) ABSTRACT

The disclosure provides in vivo methods of identifying a lipid nanoparticle that is optimized based on cellular state, delivery profile, or both for delivery into a specific single cell. The lipid nanoparticles contain an identifying DNA barcode and a VHH antibody. An agent simultaneously detects the DNA barcode, the VHH antibody, and endogenous mRNA of the cell to identify one or more viable cells having the DNA barcode and the VHH antibody at a single cell level. The cellular state of viable cells comprising the lipid nanoparticles is determined by sequencing and measuring reduced expression of one or more of an inflammatory gene, a toxicity gene, and a cell state gene compared to a cell not administered the lipid nanoparticle. Based on a favorable expression profile resulting in the cellular state, the lipid nanoparticle is selected.

### CROSS-REFERENCE TO RELATED APPLICATIONS

The present application claims the benefit of priority to U.S. Provisional Application No. 63/314,166, filed Feb. 25, 2022, the entire contents of which are hereby incorporated by reference.

### GOVERNMENT RIGHTS

This invention was made with government support under National Institutes of Health Grant UG3-TR002855 and R01DE0269. The government has certain rights in the invention.

### TECHNICAL FIELD

This disclosure relates to in vivo methods of identifying lipid nanoparticles that are specifically suitable for a cell of interest.

### BACKGROUND

In humans, lipid nanoparticles (LNPs) have delivered mRNA to antigen-presenting cells after intramuscular administration^{1, 2}, mRNA encoding Cas9 and sgRNA to hepatocytes after systemic administration³, and siRNA to hepatocytes after systemic administration⁴. These advances are tempered by clinical setbacks in which nanoparticle-mediated mRNA delivery was insufficient to treat disease⁵⁻⁷, underscoring the potential impact of LNPs with improved efficacy. To improve LNPs, scientists formulate them with chemically diverse lipids identified in vitro⁸ (cell culture) or in vivo⁹ (adult mammals). These efforts have led to LNPs that deliver mRNA to the lung, spleen, and immune cells in preclinical models¹⁰⁻¹⁵.

In addition to lipid design, clinical RNA delivery has required scientists to understand genes that influence drug delivery in vivo. In one example, LNPs were shown to deliver siRNA into hepatocytes expressing low-density lipoprotein receptor by interacting with serum apolipoprotein E in mice¹⁶. This endogenous apolipoprotein E-mediated mechanism was used in a Food and Drug Administration (FDA)-approved LNP-siRNA therapy17 and in a recent phase 1 clinical trial³. Similarly, after siRNA conjugated to modified N-Acetylgalactosamine (GalNAc) was shown to enter hepatocytes by binding asialoglycoprotein receptor (ASGPR) in mice¹⁸, GalNAc conjugates were used in FDA- and/or European Medicines Agency-approved medicines¹⁹⁻²¹ and to generate other promising clinical data^{22, 23}. Taken together, these data demonstrate that preclinical studies revealing the biological mechanism of delivery are often necessary for clinical RNA delivery. More recently, preclinical LNP-mediated mRNA delivery has been doubled²⁴ or reduced to nearly zero^{25, 26} by treating cells with small molecules that manipulate endocytic, inflammatory, or metabolic signaling, indicating that these cellular processes affect LNP delivery via yet-to-be-determined mechanisms.

Research into the biology of LNP delivery faces two key limitations, however. First, candidate genes are often identified using in vitro nanoparticle delivery. Since in vitro nanoparticle delivery does not always recapitulate in vivo nanoparticle delivery²⁷, it was reasoned that an unbiased in vivo approach could reveal alternative gene candidates. Second, the extent to which cell heterogeneity influences LNP delivery is understudied. Several lines of evidence led us to hypothesize that cells exhibit heterogeneous responses to LNPs and that these responses influence the efficiency of mRNA therapeutics. One line of evidence is that cell heterogeneity can drive metabolic²⁸ or immunological responses²⁹. Metabolic changes can increase²⁴ or decrease²⁵ LNP delivery and increasing the robustness of immunological responses decreases LNP delivery²⁶. Another line of evidence is that cells heterogeneously respond to hydrogels³⁰, which are synthetic biomaterials. Finally, LNP tropism to hepatocytes, endothelial cells, and Kupffer cells can be tuned^{10, 11, 31, 32} by modifying LNP chemistry without using targeting ligands such as antibodies, peptides, or aptamers.

An ideal way to test this hypothesis would be to measure LNP biodistribution (i.e., LNPs entering cells), functional delivery (i.e., delivered mRNA translated into functional protein), and the cellular response to LNPs, all in single cells. An ideal readout would also be generated in transcriptionally distinct single cells, thereby enabling analysis of on- and off-target delivery in any combination of cells, including rare cell types or cell types without validated fluorescence-activated cell sorting (FACS) markers. However, techniques to generate multiomic readouts of nanoparticle delivery, let alone at the single-cell level, are not well established. Accordingly, what is needed is a screening method that can be used to identify LNPs that are uniquely suitable for specific cell or cell types based on in vivo measurements.

### SUMMARY

Other features and advantages of the inventions will be apparent from the detailed description and examples that follow.

One aspect of the disclosure is directed to in vivo methods of identifying a lipid nanoparticle optimized based on cellular state and delivery profile for delivery into a specific single cell. The methods comprise:
(a) formulating multiple lipid nanoparticles having different chemical compositions, wherein each different lipid nanoparticle comprises a DNA barcode which identifies the chemical composition of the lipid nanoparticle and a VHH antibody;
(b) administering the multiple lipid nanoparticles to cells in a non-human mammal;
(c) determining the delivery profile of a lipid nanoparticle at a single cell level by: contacting the cells with an agent that simultaneously detects the DNA barcode, the VHH antibody, and endogenous mRNA of the cell to identify one or more viable cells having the DNA barcode and the VHH antibody at a single cell level based on sequencing; and identifying the DNA barcode in the one or more cells to determine the chemical composition of the delivery vehicle to correlate the chemical composition of the lipid nanoparticle with the tissue or cell type containing the nanoparticle;
(d) determining the cellular state in one or more cells at a single cell level having been administered the lipid nanoparticles by: measuring by sequencing expression of one or more of an inflammatory gene, a toxicity gene, and a cell state gene in the one or more viable cells having the DNA barcode and the VHH antibody; and identifying a cell having reduced expression of the one or more one of an inflammatory gene, a toxicity gene, and a cell state gene compared to a cell not administered the lipid nanoparticle; and
(e) selecting a lipid nanoparticle based on the delivery profile in (c) which results in the cellular state in (d).

In another aspect, the present disclosure provides in vivo methods of identifying a lipid nanoparticle optimized based on cellular state, delivery profile, or both, for delivery into a specific single cell comprising:
(a) formulating a lipid nanoparticle, wherein the lipid nanoparticle comprises an identifying DNA barcode and a VHH antibody; (b) administering a plurality of the lipid nanoparticles to cells in a non-human mammal; (c) determining the delivery profile of the lipid nanoparticle at a single cell level using steps comprising: contacting the cells with an agent that detects the DNA barcode, the VHH antibody, and endogenous mRNA of the cell to identify one or more viable cells having the DNA barcode and the VHH antibody at a single cell level based on sequencing; and identifying the DNA barcode in the one or more viable cells to determine the composition of the lipid nanoparticle to correlate the composition of the lipid nanoparticle with the tissue or cell type containing the nanoparticle; and, (d) determining the cellular state in one or more cells at a single cell level having been administered the lipid nanoparticles using steps comprising: measuring by sequencing expression of one or more of an inflammatory gene, a toxicity gene, and a cell state gene in the one or more viable cells having the DNA barcode and the VHH antibody; and identifying the lipid nanoparticle by correlating reduced expression of the one or more one of an inflammatory gene, a toxicity gene, and a cell state gene in a cell compared to a cell not administered the lipid nanoparticle with the composition of the nanoparticle, thereby identifying the lipid nanoparticle optimized based on cellular state and/or delivery profile for delivery into a specific single cell.

The disclosure also provides beads for characterizing a lipid nanoparticle having a capture sequence with a poly-T end (a PolyA binding site) and a capture sequence with a DNA barcode capture site linked to the bead. In certain embodiments, the bead is carboxyl-coated magnetic polymer bead coated with an amine reactive oligo composed of three bead barcodes (BC1-3), a sequencing adapter (GT), two linker sequences (L1-2), an UMI and Poly A binding site and/or DNA barcode binding site comprising the nucleotide sequence of SEQ ID NO: 1 or the sequence shown in FIG. 6B.

### BRIEF DESCRIPTION OF THE DRAWINGS

The summary, as well as the following detailed description, is further understood when read in conjunction with the appended drawings. For the purpose of illustrating, there are shown in the drawings' exemplary embodiments of the inventions. However, the inventions are not limited to the specific methods and compositions disclosed and the inventions are not limited to the precise arrangements and instrumentalities of the embodiments depicted in the drawings. In addition, the drawings are not necessarily drawn to scale. In the drawings:

FIG. 1A-E show a schematic of the SENT-seq methods of the disclosure.

FIG. 2A-C show in vivo multiomic single-cell readouts of transcriptome, functional LNP-mediated mRNA delivery, and LNP-mediated DNA barcode delivery. FIG. 2A shows 1-SNE of live cells sorted from the murine liver. FIG. 2B shows aVHH protein expression in the same cells, overlaid on the t-SNE plot, after administration of LNPs carrying mRNA encoding aVHH. FIG. 2C shows the most common barcode delivered by LNPs, for 24 chemically distinct LNPs, overlaid on the t-SNE plot.

FIG. 3A-3C show cell subset differently uptake LNPs. FIG. 3A shows normalized barcode distribution profiles for endothelial cells, violin plots representing the spread of normalized barcode distribution profiles, and FIG. 313 shows the accompanying plots for aVHH expression profiles for endothelial cells. The same normalized barcode distribution profiles are also shown for Kupffer cells (FIG. 3C) and Hepatocytes (FIG. 3D). Cell types with narrow distributions are characterized by narrow unimodal peaks of low normalized barcode delivery. Cell types with wide distributions are characterized by wide peaks or bimodal peaks of low and high normalized barcode delivery.

FIG. 4A-H show that endothelial cell subtypes have transcriptional differences that may dictate LNP mediated mRNA delivery. FIG. 4A shows a schematic of liver vessel morphology. FIG. 4B is a dot map showing expression levels of 18 important genes in hepatic endothelial cell differentiation. FIG. 4C and FIG. 4D are volcano plot of differentially expressed genes in EC1 as compared to EC3 (FIG. 4C) and EC2 as compared to EC3 (FIG. 4D). FIG. 4E shows an explanation of differential analysis comparison between EC clusters to identify genes. FIG. 4F shows a Venn diagram of differentially expressed genes found in EC2 as compared to EC3 and EC1 after separation based on aVHH expression. FIG. 4G shows STRING analysis of the 19 differentially expressed genes found in aVHH positive cells in endothelial cell cluster 1 and 2. FIG. 4H shows a dot map of the expression levels of differentially expressed genes with significant interactions.

FIG. 5A-N show chemically distinct LNPs exhibit different tropism within the liver microenvironment. Each LNP is formulated to contain a distinct DNA barcode, which were able to be mapped onto single cells. LNP barcode counts are represented in each cell cluster as either the average of barcode counts for all single cells within a cluster (FIG. 5A), or the sum of barcode counts for all single cells within a cluster (FIG. 5B). The three negative control naked barcodes are represented by a “*”(FIG. 5C-F). The distribution of LNPs, identified based on their DNA barcode, overlaid on a t-SNE of 17 distinct cell subsets, shown alongside each LNPs composition as shown in FIG. 5G-J. FIG. 5K-N show the distribution of cell types within cells that contain a particular LNP. The aVHH to barcode ratio for all four LNPs in all cell types where those LNPs are found.

FIG. 6A and FIG. 6B show the orthogonal capture sequences to generate tunable multiomic readouts. FIG. 6A shows the barcode structure for screening of LNPs; barcodes contain different regions highlighted above. FIG. 6B shows the beads used for microwell-based single-cell RNA sequencing were modified to include both an mRNA binding site and a barcode binding site.

FIG. 7A-D show that SENT-seq utilizes orthogonal capture sequences to generate tunable multiomic readouts. FIG. 7A shows the compounds included in LNP formulation as well as molar ratios screened. LNPs were formulated so that they contained an ionizable lipid, PEG-lipid, phospholipid, and cholesterol. Molar ratios of LNP constituents are shown for each LNP. LNP characteristics such as (LNP diameter (FIG. 7B), polydispersity index (FIG. 7C), and encapsulation efficiency (FIG. 7D) are shown for all individual pooled LNPs.

FIG. 8A and FIG. 8B show in vivo multiomic single-cell readouts of transcriptome, functional LNP-mediated mRNA delivery, and LNP-mediated DNA barcode delivery. Representative in vivo gating strategies for liver cell populations (FIG. 8A) and aVHH+ cells within those cell populations (FIG. 8B).

FIG. 9A-D show in vivo multiomic single-cell readouts of transcriptome, functional LNP-mediated mRNA delivery, and LNP-mediated DNA barcode delivery. FIG. 9A is UMAP projection showing the distribution of hepatic clusters from mouse livers treated with LNP pool and PBS. The gene expression analysis in FIG. 9B shows 17 distinct cell clusters that contain different transcriptomic profiles. FIG. 9C shows the percentage of each hepatic cell cluster. FIG. 9D shows background levels of aVHH expression in control mice treated with 1×PBS represent a stringent aVHH expression cutoff.

FIG. 10A-H show in vivo multiomic single-cell readouts of transcriptome, functional LNP-mediated mRNA delivery, and LNP-mediated DNA barcode delivery. Percentage of aVHH+ cells determined using single-cell RNA sequencing (scRNA-seq), with background levels found in control mice subtracted, in endothelial cell subsets (FIG. 10A), Kupffer cell subsets (FIG. 10B), hepatocyte subsets (FIG. 10C), B and T cells (FIG. 10D), Ito cell subsets (FIG. TOE), and cholangiocytes and erythroid cells (FIG. 10F). FIG. TOG shows the percentage of aVHH+ cells in populations analyzed using flow cytometry. FIG. 10H shows the comparison of the percentage of aVHH+ cells in the whole hepatic population determined using flow cytometry and scRNA-seq. Statistical analyses were conducted using a one-way factor ANOVA with Sidak's multiple comparison test for every cell population that had multiple subtypes within that population as well as for the flow cytometry and flow cytometry versus scRNA-seq comparisons. ns (p>0.05, not shown), * (p<0.05), ** (p<0.01), *** (p<0.001).

FIG. 11 shows in vivo multiomic single-cell readouts of transcriptome, functional LNP-mediated mRNA delivery, and LNP-mediated DNA barcode delivery. Distribution of LNP barcodes in mice treated with LNP pool. As noted, naked barcodes, the negative control, each made up less than 0.5% of barcodes delivered to hepatic cells.

FIG. 12A-D show that cell subsets differentially uptake LNPs. aVHH expression profiles and violin plots representing the spread of aVHH expression are shown for Kupffer cells (FIG. 12A), hepatocytes (FIG. 12B), Ito cells (FIG. 12C), and B cells (FIG. 12D).

FIG. 13A and FIG. 13B show that cell subsets differentially uptake LNPs. Normalized barcode distribution profiles and violin plots representing the spread of normalized barcode distribution profiles shown for Ito cells (FIG. 13A) and B cells (FIG. 13B).

FIG. 14A-D show that other cell subtypes have transcriptional differences that may not affect mRNA delivery. Differential analysis of hepatocyte (FIG. 14A) and Kupffer cells (FIG. 14B) of low-delivery clusters (Hep2 and KC3) as compared to high-delivery clusters (Hep1/3 and KC2/1). Venn diagram comparison of upregulated genes in hepatocyte (FIG. 14C) and Kupffer cell clusters (FIG. 14D) after segregation based on aVHH− and aVHH+ expression.

FIG. 15 shows that chemically distinct LNPs exhibit different tropism within the liver microenvironment. tSNE plots showing normalized barcode expression in each cell type for each LNP in the pool. Naked barcodes (*) had low normalized expression across all cells.

FIG. 16 shows that chemically distinct LNPs exhibit different tropism within the liver microenvironment. tSNE plots showing aVHH expression in each cell type for each LNP in the pool. Naked barcodes (*) had low aVHH expression across all cells.

FIG. 17 shows that chemically distinct LNPs exhibit different tropism within the liver microenvironment. tSNE plots showing the ratio of aVHH expression to barcode expression in each cell type for each LNP in the pool.

FIG. 18 shows that chemically distinct LNPs exhibit different tropism within the liver microenvironment. When confirmed with our traditional sequencing methods, LNP-3, LNP-7, LNP-10, and LNP-12 were among LNPs with the highest normalized barcode expression. All other LNPs are shown in gray, and naked barcodes are shown in black.

FIG. 19A-D show the cKK-E15 synthesis pathway. Synthesis pathway for cKK-E15 and intermediates, used as an ionizable lipid for the LNP screen as shown in FIGS. 19A and 19B. FIG. 19C shows the 1H-NMR and FIG. 19D shows the ¹³C NMR measurements for cKK-E15.

FIG. 20 shows the mouse weights for experiments. Changes in weight for mice treated with the control, 1× PBS, did not differ from changes in weight for mice treated with the LNP pool.

### DETAILED DESCRIPTION

In the following detailed description of the illustrative embodiments, reference is made to the accompanying drawings that form a part hereof. These embodiments are described in sufficient detail to enable those skilled in the art to practice the invention, and it is understood that other embodiments may be utilized, and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense.

An ideal drug delivery readout would measure LNP biodistribution (i.e., LNPs entering cells), functional delivery (i.e., delivered mRNA translated into functional protein), and the cellular response to LNPs. Moreover, it would generate these data in single cells, alongside the transcriptome of each cell, thereby creating two key advantages. First, measuring delivery in transcriptionally defined single cells makes it possible to quantify rare cell types, cell subtypes, and cells defined by a specific gene of interest (e.g., a transcription factor). In addition, since these assays do not require FACS markers, this approach could enable high-throughput screens with detailed on-/off-target delivery in animals such as non-human primates (NHPs), which do not have established FACS panels for all desired cell types. Second, since delivery is measured alongside cell response to the delivery vehicle, this approach could lead to novel insights regarding the genes and pathways that affect drug delivery. To that end, this disclosure provides for in vivo method of identifying a lipid nanoparticle that has been optimized based on cellular state and delivery profile for delivery into a specific single cell.

The in vivo method of the disclosure are unique in that they allow detection of a lipid nanoparticle in a specific cell and the response of that specific cell. The methods of disclosure thus function at the single cell level. The in vivo methods allow for simultaneous detection of the lipid nanoparticle and the cell's response by using sequencing.

The Single-Cell Nanoparticle Targeting-sequencing (SENT-seq) methods of the disclosure use uses (i) DNA barcodes to quantify how many chemically distinct LNPs target cells in the same animal, (ii) DNA tagged antibodies to measure the functional translation of LNP-delivered mRNA, and (iii) RNA sequencing to measure the transcriptome all with single-cell resolution. This disclosure uniquely provides a high-throughput in vivo drug delivery assay with single-cell resolution as well as the simultaneous determining (i) and (i). By using SENT-seq to quantify how many LNPs deliver to 17transcriptionally defined cell subtypes within the liver, the inventors have generated a newly detailed readout of on- and off-target delivery.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### In Vivo Methods of Identifying Optimized Lipid Nanoparticles

One aspect of the disclosure is directed to in vivo methods of identifying a lipid nanoparticle that has been optimized based on cellular state and delivery profile for delivery into a specific single cell. In certain embodiments, the methods identify lipid nanoparticles that do not induce toxicity or immune activation, such as e.g. during the screening method.

The methods of disclosure use lipid nanoparticles having different chemical compositions. Each of the different lipid nanoparticle comprises a DNA barcode which identifies the chemical composition of the lipid nanoparticle and a VHH antibody. These lipid nanoparticles are administered to mammalian cells in vivo.

The methods of the disclosure also include determining the cellular state in one or more cells at a single cell level that have administered the lipid nanoparticle. In certain embodiments, the methods include simultaneously determining the cellular state in one or more cells at a single cell level that have administered the lipid nanoparticle.

The determining the cellular state is achieved by measuring by sequencing expression of one or more of an inflammatory gene, a toxicity gene, and/or a cell state gene in the one or more viable cells that have been administered the lipid nanoparticle. These cells are identified based on the presence of the DNA barcode and the VHH antibody. Based on comparing the cell state and a nanoparticles, it is possible to identify which nanoparticle is optimal for delivery into the cell. When the cells have reduced expression of the one or more one of an inflammatory gene, a toxicity gene, and a cell state gene compared to a cell not administered the lipid nanoparticle are cells, the nanoparticle is optimal for delivery into the cell. In one embodiment, the method includes measuring by sequencing expression of one or more of an inflammatory gene, a toxicity gene, and a cell state gene in the one or more viable cells.

In certain embodiments, the methods also include measuring by sequencing the expression of the same one or more of an inflammatory gene, a toxicity gene, and a cell state gene in a cell that has not been contacted in the lipid nanoparticles. In other embodiments, the methods include provide the previous measurements of the same one or more of an inflammatory gene, a toxicity gene, and a cell state gene in a cell that has not been contacted in the lipid nanoparticles.

In certain embodiments, the methods include measuring the expression of at least one inflammatory gene, at least one toxicity gene, and at least one cell state gene. Alternatively, the methods include measuring (i) one or more, two or more, three or more, four or more, five or more, six or more, seven or more, eight or more, nine or more, ten or more, eleven or more, twelve or more inflammatory genes; (ii) one or more, two or more, three or more, four or more, five or more, six or more, seven or more, eight or more, nine or more, ten or more, eleven or more, twelve or more toxicity genes; and/or (iii) one or more, two or more, three or more, four or more, five or more, six or more, seven or more, eight or more, nine or more, ten or more, eleven or more, twelve or more cell state genes.

In certain embodiments, the inflammatory gene is Apoa2, CD163, Dnajb9, Traf3, and/or combinations thereof. In other embodiments, the toxicity gene is Gsk3b, Rpto, Dnm1, Casp3, and/or combinations thereof. In alternate embodiments, the cell state gene is CDk9, Rdx, Ldir, Atm, and/or combinations thereof. In yet another embodiment, the inflammatory gene is Apoa2, CD163, Dnajb9, Traf3, and/or combinations thereof, the toxicity gene is Gsk3b, Rpto, Dnm1, Casp3, and/or combinations thereof, and/or the cell state gene is CDk9, Rdx, Ldir, Atm, and/or combinations thereof.

In addition to measuring expression of one or more of an inflammatory gene, a toxicity gene, and/or a cell state gene, the methods may include measuring by sequencing expression of one or more gene indicative of endocytosis. In certain embodiments, increased expression of one more gene indicative of endocytosis when compared to a cell not administered the lipid nanoparticle is indicative of a lipid nanoparticle having improved uptake in the cell. In certain embodiments, increased expression of one more gene indicative of endocytosis when compared to a cell not administered the lipid nanoparticle is indicative of a lipid nanoparticle having improved uptake in the cell.

The methods of the disclosure do not comprise measuring protein levels. In certain embodiments, the methods include quantifying the lipid nanoparticles in the single cell (i.e. at the single cell level). In other embodiments, the methods simultaneously identify the DNA barcode in the cell and measure expression (by sequencing) of the one or more of an inflammatory gene, a toxicity gene, and a cell state gene in the viable cells having the DNA barcode and the VHH antibody.

Additional examples of inflammatory genes that may be measured in the methods of the invention are shown in Table 1 below. In certain embodiments of the disclosure, the methods of the invention may measure expression of one or more, two or more, three or more, four or more, five or more, six or more, seven or more, eight or more, nine or more, ten or more, eleven or more, twelve or more of the inflammatory genes shown in Table 1 below.

TABLE 1
Examples of inflammatory genes for use in the methods
Gene ID
CCL2
CCL3
ccl7
ccl12
cxcl1
cxcl2
IL-1b
GM-CSF
IL-6
cxcl1
cxcl2
cxcl5
cxcl10
ccl2
ccl3
ccl4
ccl7
ccl12
csf2
csf3
TLR3
TLR7
TLR8
NFKB
caspase 1
IRF3
IRF7
PKR
PAS
Abcf1
Ace
Ackr1
Ackr2
Ackr3
Ackr4
Acox1
Acsl1
Acsl3
Acsl4
Acvr1
Adar
Adgre5
Adgrg3
Adora2a
Ager
Agt
Ahr
Aif1
Aim2
Akt1
Akt2
Akt3
Alas1
Alox12
Alox15
Alox5
Alox5ap
Alpk1
Alpl
Anpep
Ap1g1
Ap1m1
Ap1s2
Apbb1ip
Apex1
Apol6
App
Arrb2
Atf2
Atf4
Atf6
Atg10
Atg12
Atg13
Atg3
Atg4a
Atg7
Atm
Atp6ap2
Atp6v0d1
Atp6v1b2
Batf
Bax
Bcl2
Bcl2l1
Bcl3
Bcl6
Bcr
Bdkrb1
Bdkrb2
Becn1
Blk
Bnip3
Bpi
Bst2
C1qbp
C2
C3
C3ar1
C5ar1
Calm1
Cap1
Card11
Casp1
Casp3
Casp4
Casp8
Cbfb
Cbl
Cblb
Ccl1
Ccl11
Ccl12
Ccl17
Ccl19
Ccl2
Ccl20
Ccl21d
Ccl22
Ccl24
Ccl25
Ccl26
Ccl27b
Ccl28
Ccl3
Ccl4
Ccl5
Ccl6
Ccl7
Ccl8
Ccl9
Ccnc
Ccr1
Ccr10
Ccr1l1
Ccr2
Ccr3
Ccr4
Ccr5
Ccr6
Ccr7
Ccr8
Ccr9
Ccrl2
Cd14
Cd163
Cd19
Cd2
Cd209e
Cd22
Cd244a
Cd247
Cd27
Cd274
Cd276
Cd28
Cd36
Cd38
Cd3d
Cd3e
Cd3g
Cd4
Cd40
Cd40lg
Cd44
Cd59a
Cd6
Cd68
Cd69
Cd70
Cd79a
Cd79b
Cd80
Cd81
Cd84
Cd86
Cd8a
Cd8b1
Cd96
Cdh1
Cdk4
Ceacam3
Cebpb
Cfd
Cflar
Cgas
Chuk
Cklf
Cmklr1
Cpa3
Crcp
Creb1
Crebbp
Crk
Crp
Csf1
Csflr
Csf2
Csf2ra
Csf2rb
Csf3
Csf3r
Ctla4
Ctsa
Ctsg
Ctsl
Ctss
Ctsw
Ctsz
Cul1
Cx3cl1
Cx3cr1
Cxcl1
Cxcl10
Cxcl12
Cxcl13
Cxcl14
Cxcl15
Cxcl16
Cxcl17
Cxcl2
Cxcl3
Cxcl5
Cxcl9
Cxcr1
Cxcr2
Cxcr3
Cxcr4
Cxcr5
Cxcr6
Cyp2e1
Cystm1
Ddah2
Ddit3
Ddost
Ddx5
Ddx58
Defb14
Derl1
Dhx58
Diablo
Dnaja2
Dnajc10
Dtx3l
Dysf
Ebi3
Egln1
Eif2ak2
Eif2ak3
Eif3f
Elane
Entpd1
Eomes
Ep300
Ephx2
Ern1
Ets1
Evl
F5
Fas
Fasl
Fbxo6
Fcer1a
Fcgr1
Fcgr2b
Fcgr4
Fcgrt
Fcrlb
Fgr
Fos
Foxo1
Foxp3
Fpr1
Fpr2
Furin
Fyn
Gab2
Gadd45b
Gata3
Gba
Gbp2
Gbp3
Gbp5
Gca
Gk
Gla
Glb1
Gns
Gpx7
Gsk3b
Gstm4
Gucy 1a1
Gucy 1b1
Gusb
Gzma
Gzmb
Gzmc
Gzmd
Gzme
Gzmf
Gzmg
Gzmk
Gzmm
Gzmn
H2-Ab1
H2-D1
H2-DMa
H2-DMb1
H2-DMb2
H2-Eb1
H2-K1
H2-M3
H2-Ob
H2-Q1
H2-Q10
H2-Q2
H2-T23
Hamp
Havcr2
Hc
Hck
Hcst
Hdc
Hk3
Hlx
Hmgb1
Hmox1
Hpgd
Hprt
Hsd11b1
Hsp90aa1
Hsp90ab1
Hsp90b1
Hspb1
Icos
Icosl
Ido1
Ifi27
Ifi35
Ifi44
Ifih1
Ifit1
Ifit2
Ifit3
Ifitm1
Ifitm2
Ifitm3
Ifna12
Ifna2
Ifna4
Ifnar1
Ifnar2
Ifnb1
Ifne
Ifng
Ifngr1
Ifngr2
Ifnk
Ifnl2
Ifnlr1
Ifnz
Igfbp7
Ikbkb
Ikbke
Ikbkg
Il10
Il10ra
Il10rb
Il11
Il11ra1
Il12a
Il12b
Il12rb1
Il12rb2
Il13
Il13ra1
Il13ra2
Il15
Il15ra
Il16
Il17a
Il17b
Il17c
Il17d
Il17f
Il17ra
Il17rb
Il17rc
Il17rd
Il17re
Il18
Il18bp
Il18r1
Il18rap
Il19
Il1a
Il1b
Il1f10
Il1r1
Il1r2
Il1rap
Il1rapl1
Il1rapl2
Il1rl1
Il1rl2
Il1rn
Il2
Il20
Il20ra
Il20rb
Il21
Il21r
Il22
Il22ra1
Il22ra2
Il23a
Il23r
Il24
Il25
Il27
Il27ra
Il2ra
Il2rb
Il2rg
Il3
Il31
Il31ra
Il33
Il34
Il36a
Il36b
Il36g
Il36rn
Il3ra
Il4
Il4ra
Il5
Il5ra
Il6
Il6ra
Il6st
Il7
Il7r
Il9
Il9r
Irak1
Irak3
Irak4
Irf1
Irf3
Irf4
Irf7
Irf9
Isg15
Itgae
Itgal
Itgam
Itgax
Itgb2
Itgb7
Itk
Itln1
Itm2c
Itpr3
Jak1
Jak2
Jak3
Jaml
Jun
Junb
Kdm6b
Kir3dl1
Klra1
Klrb1
Klrc1
Klrd1
Klrk1
Kpnb1
Kras
Lag3
Lamp1
Lamp2
Lamp3
Lancl1
Lat
Lat2
Lax1
Lck
Lcn2
Lcp1
Lcp2
Ldhb
Lef1
Lgals3
Lif
Lilra5
Lilra6
Limk2
Litaf
Lrg1
Lrrk2
Lta4h
Ltb
Ltbr
Ltc4s
Ltf
Ly96
Lyn
Lyz2
Maf
Mafb
Map1lc3a
Map2k2
Map2k3
Map2k4
Map2k7
Map3k1
Map3k3
Map3k5
Map3k7
Map3k8
Mapk1
Mapk13
Mapk14
Mapk8
Mapk9
Mapkapk2
Marcks
Marco
Mavs
Mcl1
Mcoln1
Mdfic
Mefv
Mgam
Mif
Mknk1
Mlkl
Mme
Mmp9
Mrc1
Mrps7
Ms4a1
Ms4a2
Ms4a7
Msra
Mtor
Mvp
Myc
Myd88
Nae1
Nampt
Ncf1
Ncf2
Ncf4
Ncr1
Ndufs8
Neo1
Neu1
Nfat5
Nfatc1
Nfatc2
Nfatc3
Nfatc4
Nfe2l2
Nfkb1
Nfkb2
Nfkbia
Ngly 1
Nkg7
Nlrc4
Nlrc5
Nlrp1a
Nlrp3
Nmt1
Nod1
Nod2
Nos2
Notch1
Nox1
Npc2
Nras
Nrde2
Nt5e
Ntng2
Oas1a
Oas2
Oas3
Oasl1
Oaz1
Os9
Osm
P2rx7
Pak1
Panx1
Parp1
Parp9
Pdcd1
Pdcd1lg2
Pdhb
Pecam1
Peli1
Peli2
Pfkfb3
Pgk1
Pik3ap1
Pik3c3
Pik3ca
Pik3cb
Pik3cd
Pik3cg
Pik3r3
Pik3r4
Pik3r5
Pik3r6
Pirb
Plat
Plau
Plaur
Plcg1
Plcg2
Plek
Plekha1
Plg
Plin4
Plscr1
Plscr2
Pnoc
Ppia
Prop
Prdm1
Prf1
Prkca
Prkcd
Prkcq
Prkcsh
Psap
Psmb10
Psmb8
Psmb9
Pstpip1
Ptger2
Ptger4
Ptgs2
Ptk2b
Ptpn4
Ptpn6
Ptprc
Pxn
Pycard
Rab31
Rab5c
Rab7
Rac2
Rack1
Raf1
Rasgrp1
Rasgrp4
Rb1cc1
Rbck1
Rbpj
Rel
Rela
Relb
Rgma
Rhog
Ripk1
Ripk2
Ripk3
Rnasel
Rnf114
Rnf135
Rnf31
Rps6ka1
Rps6ka3
Rps6kb1
Rsad2
Runx3
Samhd1
Scarb2
Sdha
Sele
Selenos
Sell
Sem1
Serpina1a
Sh2d1a
Sigirr
Sirpa
Slc11a1
Slc2a3
Smad3
Smad4
Smad5
Socs1
Socs3
Sod1
Sod2
Sort1
Sp1
Sp100
Spi1
Spib
Ssr1
Stat1
Stat2
Stat3
Stat4
Stat5a
Stat5b
Stat6
Sting1
Stk11ip
Strap
Stt3b
Sugt1
Syk
Tab1
Tab2
Tank
Tap1
Tap2
Tbk1
Tbp
Tbx21
Tbxas1
Tcirg1
Tcl1
Tcn2
Tgfb1
Tgfb2
Tgfb3
Tgfbr2
Thbs1
Thop1
Ticam1
Tifa
Tigit
Timp2
Tirap
Tln1
Tlr1
Tlr2
Tlr3
Tlr4
Tlr5
Tlr6
Tlr7
Tlr8
Tlr9
Tmem140
Tmprss2
Tnf
Tnfrsf10b
Tnfrsf17
Tnfrsf18
Tnfrsf1a
Tnfrsf25
Tnfrsf4
Tnfrsf9
Tnfsf10
Tnfsf13b
Tnfsf18
Tnfsf4
Tnfsf9
Tollip
Tpp1
Tpsb2
Traf2
Traf3
Traf6
Tram1
Trat1
Trim21
Trim25
Trim26
Trim33
Trim56
Trim6
Txk
Txn1
Txnip
Tyk2
Tyrobp
Uba52
Ube2l6
Ube2n
Ulk1
Ulk2
Vamp3
Vcam1
Vegfa
Vrk3
Vsir
Vwf
Was
Wipi1
Xaf1
Xbp1
Xcl1
Xcr1
Ywhaq
Zap70
Zbp1

Examples of toxicity genes that may be used in the methods of the invention are shown in Table 2 below. In certain embodiments of the disclosure, the methods of the invention may measure expression of one or more, two or more, three or more, four or more, five or more, six or more, seven or more, eight or more, nine or more, ten or more, eleven or more, twelve or more of the toxicity genes shown in Table 2 below.

TABLE 2
Examples of toxicity genes for use in the methods

| Gene ID | Gene ID | Gene ID | Gene ID | Gene ID | Gene ID |
|---|---|---|---|---|---|
| CD95 | Akt1 | Ctse | Gusb | Ncoa7 | Trp53 |
| TNFR1 | Apoa5 | Cyb561d1 | Herpud1 | Nos2 | Txnl4b |
| CASP8 | Apof | Cyld | Hmox1 | Nqo1 | Uhrf1 |
| CASP3 | Arrdc3 | Cyp1a2 | Hpn | Nr5a2 | Wipi1 |
| APAF-1 | Asah1 | Cyp2b10 | Hprt | Parp2 | |
| CASP9 | Asb1 | Cyp2c40 | Hsf1 | Pdyn | |
| GrA | Asns | Cyp2c66 | Hspa12a | Polr1b | |
| GAAD | Atf3 | Cyp2d9 | Hspa1b | Ppara | |
| CASP12 | Atm | Dnajb1 | Hspa4 | Pvr | |
| Abca1 | Atp8b1 | Dnajb9 | Hspa5 | Rad51 | |
| Abcb4 | Bcl2 | Eno2 | Id1 | Rdx | |
| Abcc2 | Bcl2l1 | Ercc2 | Il6 | Rplp0 | |
| Abcc3 | Brca1 | Fasl | Inhbe | Slc25a25 | |
| Abcf1 | Casp8 | Fasn | Ldha | Slc2a3 | |
| Acadm | Casp9 | Fhl2 | Lpl | Slc7a11 | |
| Acadvl | Cd36 | Gclm | Lss | Sod1 | |
| Acox1 | Cdkn1a | Gpx1 | Mag | Sod3 | |
| Adh1 | Cpt1a | Gpx2 | Mdm2 | Srebf1 | |
| Adm2 | Cpt1b | Grb2 | Metap2 | Trib3 | |
| Akr1c21 | Cpt2 | Gsta3 | Mki67 | Trim10 | |

Examples of suitable cell state genes that may be used in the methods of the invention are shown in the table below. In certain embodiments of the disclosure, the methods of the invention may measure expression of one or more, two or more, three or more, four or more, five or more, six or more, seven or more, eight or more, nine or more, ten or more, eleven or more, twelve or more of the cell state genes shown in Table 3 below.

TABLE 3
Examples of cell state genes for use in the methods

| Gene ID | Gene ID | Gene ID | Gene ID | Gene ID |
|---|---|---|---|---|
| MTORC1 | CD163 | FOXP3 | LAMTOR4 | NOS2 |
| Akt | CD180 | IL10 | LAMTOR5 | NOS3 |
| PTEN | CD19 | IL2 | LAT | NOX1 |
| PIK3CA | CD209 | IL21R | LCK | NOX3 |
| RHEB | CREB3L3 | IL2RA | LDHA | NOX4 |
| BMP2 | CYP1A1 | IL4 | LY86 | PIK3C2A |
| Fos | CYP1A2 | IL4I1 | LY96 | PIK3CA |
| BTG2 | CYP1B1 | IL6 | MAP1LC3B | PIK3CB |
| LY6C1 | CYP4A11 | IL7 | MAP2K1 | PIK3CD |
| IL-17 | CYP4A22 | ITGA1 | MAP2K2 | PIK3R1 |
| STAT3 | CYP8B1 | ITGA11 | MAP2K3 | PIK3R2 |
| HIF-1a | ENO1 | ITGAM | MAP3K12 | PIK3R3 |
| CD244 | ENO3 | ITGB1 | MAPK1 | PIK3R4 |
| CD247 | EXO1 | ITGB2 | MAPK8 | RPTOR |
| CD27 | EZH2 | ITGB5 | MAPK8IP1 | SOX2 |
| CD274 | FABP5 | LAMA4 | MAPKAP1 | TLR10 |
| CD276 | FAH | LAMB1 | NFKB1 | TLR2 |
| CD28 | FAHD1 | LAMC1 | NFKB2 | TLR4 |
| CD14 | FOXM1 | LAMTOR2 | NOS1 | TLR7 |

Examples of suitable endocytosis genes that may be used in the methods of the invention are shown in Table 4 below. In certain embodiments of the disclosure, the methods of the invention may measure expression of one or more, two or more, three or more, four or more, five or more, six or more, seven or more, eight or more, nine or more, ten or more, eleven or more, twelve or more of the endocytosis genes shown below.

TABLE 4
Examples of endocytosis genes for use in the methods

| Gene ID | Gene ID | Gene ID |
|---|---|---|
| SREBF2 | MYLIP | CAV3 |
| SREBF1 | NPC1 | CLTA |
| LDLR | RAB7 | CLTB |
| APOE | RAB6 | CLTC |
| PCSK9 | RAB4 | DNM1 |
| VLDLR | RAB5A | THBS2 |
| LDLRAP1 | RAB9 | TF |
| DAB2 | RAB11 | APOA2 |
| APOA1 | LAMP1 | APOA4 |
| APOB | CD63 | APOC2 |
| D36 | EEA1 | APOC3 |
| CAV2 | CAV1 | APOM |

In certain embodiments of the methods, the agent that simultaneously detects the DNA barcode, the VHH antibody, and the endogenous mRNA of the cells is a bead having a capture sequence with a poly-T end (a PolyA binding site) and a capture sequence with a DNA barcode capture site. In these beads, the DNA barcode capture site is capable of binding a universal sequence found in all of the DNA barcodes. Furthermore, in certain embodiments, the poly-T end detects the VHH antibody and endogenous mRNA of the cell. In other embodiments, the bead is a carboxyl-coated magnetic polymer bead. In other embodiments, the agent is a bead as described below are as used in the Examples.

One embodiment of the disclosure is an in vivo method of identify a lipid nanoparticle that has been optimized based on cellular state and delivery profile for delivery into a specific single cell which includes the steps of:
(a) formulating multiple lipid nanoparticles having different chemical compositions, which each different lipid nanoparticle having a DNA barcode which identifies the chemical composition of the lipid nanoparticle and a VHH antibody;
(b) administering the multiple lipid nanoparticles to cells in a non-human mammal;
(c) determining the delivery profile of a lipid nanoparticle at a single cell level by contacting the cells with an agent that simultaneously detects the DNA barcode, the VHH antibody, and endogenous mRNA of the cell to identify one or more viable cells having the DNA barcode and the VHH antibody at a single cell level based on sequencing; and identifying the DNA barcode in the one or more cells to determine the chemical composition of the delivery vehicle to correlate the chemical composition of the lipid nanoparticle with the tissue or cell type containing the nanoparticle;
(d) determining the cellular state in one or more cells at a single cell level having been administered the lipid nanoparticles by measuring by sequencing expression of one or more of an inflammatory gene, a toxicity gene, and a cell state gene in the one or more viable cells having the DNA barcode and the VHH antibody and identifying a cell having reduced expression of the one or more one of an inflammatory gene, a toxicity gene, and a cell state gene compared to a cell not administered the lipid nanoparticle; and
(e) selecting a lipid nanoparticle based on the delivery profile in (c) which results in the cellular state in (d).

In one embodiment, the method includes measuring the expression of one or more of an inflammatory gene, a toxicity gene, and a cell state gene in a cell that has not been contacted in the lipid nanoparticles. In another embodiment, the method includes measuring the expression of at least one inflammatory gene, at least one toxicity gene, and at least one cell state gene.

In certain embodiments, the inflammatory gene measured in the methods is selected from the group consisting of Apoa2, CD163, Dnajb9, Traf3, and combinations thereof. In other embodiments, the inflammatory gene is one or more gene shown in Table 1. In other embodiments, the toxicity gene is selected from the group consisting of Gsk3b, Rpto, Dnm1, Casp3, and combinations thereof. In additional embodiments, the toxicity gene is one or more gene shown in Table 2. In yet further embodiments, the cell state gene is selected from the group consisting of CDk9, Rdx, Ldir, Atm, and combinations thereof. In yet alternate embodiments, the cell state gene is one or more gene shown in Table 3.

In certain embodiments, the method includes measuring expression of one or more gene indicative of endocytosis and measuring the expression of one or more of an inflammatory gene, a toxicity gene, and a cell state gene in the viable cells having the DNA barcode and the VHH antibody. In some embodiments, increased expression of one more gene indicative of endocytosis when compared to a cell not administered the lipid nanoparticle is indicative of a lipid nanoparticle having improved uptake in the cell. In other embodiments, the one of more gene indicative of endocytosis is one or more gene shown in Table 4.

In one embodiment, the method identifies lipid nanoparticles that do not induce toxicity or immune activation during the screening method. In another embodiment, the method comprises simultaneously identifying the DNA barcode in the cell and measuring expression of the one or more of an inflammatory gene, a toxicity gene, and a cell state gene in the viable cells having the DNA barcode and the VHH antibody.

In certain embodiments of the method the agent that simultaneously detects the DNA barcode, the VHH antibody, and the endogenous mRNA of the cells is a bead having a capture sequence with a poly-T end (a PolyA binding site) and a capture sequence with a DNA barcode capture site. In one embodiment, the DNA barcode capture site is capable of binding a universal sequence found in all of the DNA barcodes. In another embodiment, the poly-T end detects the VHH antibody and endogenous mRNA of the cell.

In yet another embodiment, the bead is a carboxyl-coated magnetic polymer bead. In certain embodiments, the method also includes administering a lipid nanoparticle identified by the method, which contains a therapeutic agent, to a patient in need of the therapeutic agent such as e.g. a cancer patient.

### Beads for Characterizing a Lipid Nanoparticles

The disclosure also provides beads for characterizing a lipid nanoparticle having a capture sequence with a poly-T end (a PolyA binding site) and a capture sequence with a DNA barcode capture site linked to the bead.

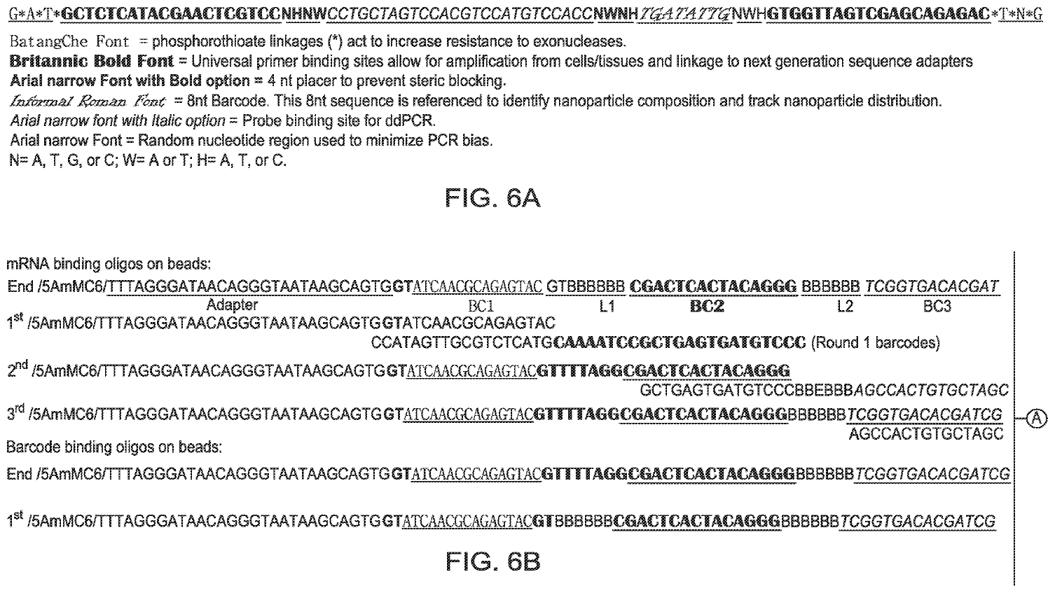

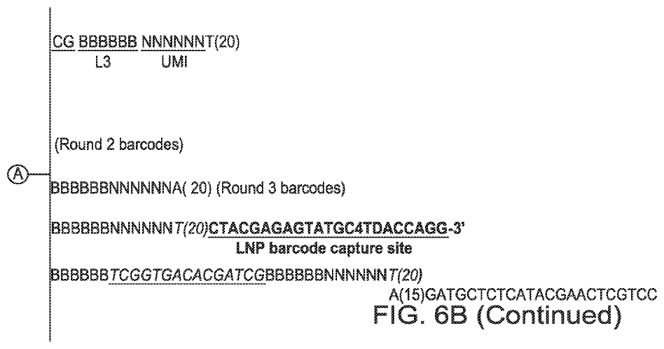

The DNA barcode capture site of the beads is capable of binding a universal sequence DNA sequence found in DNA barcodes for lipid nanoparticles. In certain embodiments, the DNA barcode capture site of the beads comprises the LNP barcode capture site shown in FIG. 6B.

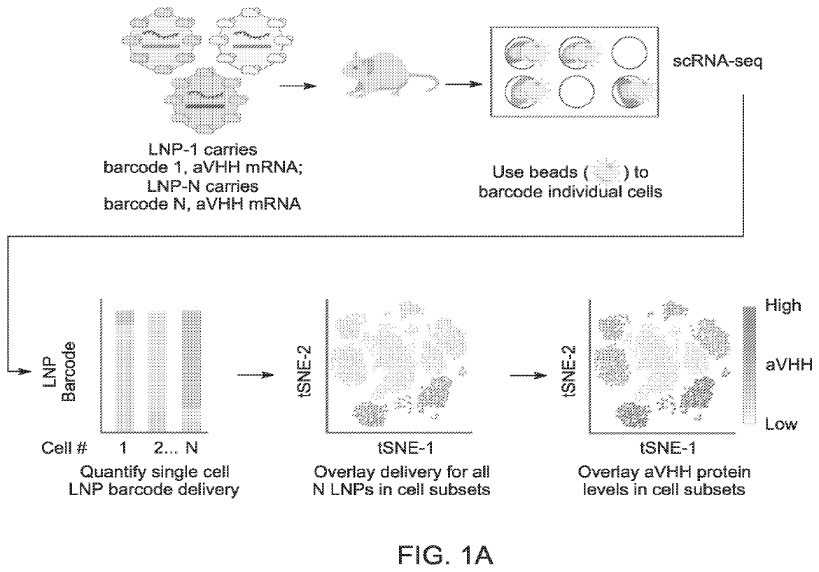

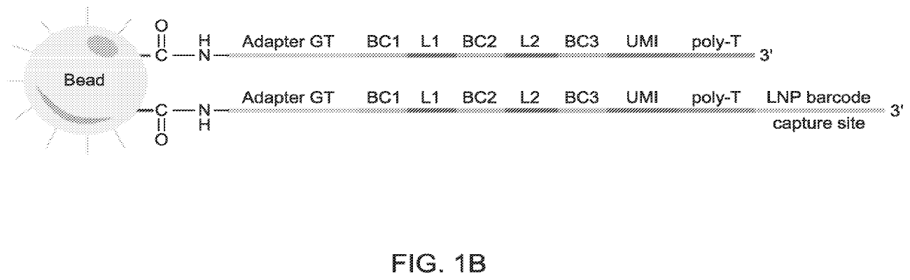

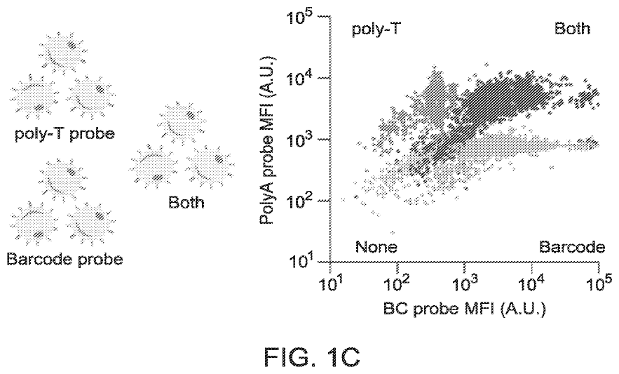

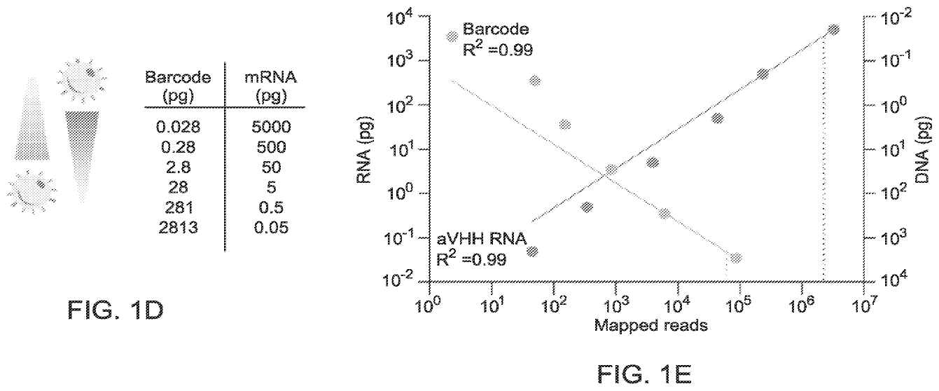

In certain embodiments, the bead has a structure as shown in FIG. 1B. In other embodiments, the bead recognizes a barcode shown in FIG. 6A or the capture sequence with the poly-T end and a capture sequence with a DNA barcode capture site linked to the bead have the sequences shown in FIG. 68 or an LPN barcode capture site having SEQ ID NO: 1 (TAC GAG AGT ATG CCT GAGC AGG).

In certain embodiments, the bead is carboxyl-coated magnetic polymer bead coated with an amine reactive oligo composed of three bead barcodes (BC1-3), a sequencing adapter (GT), two linker sequences (L1-2), an UMI and PolyA binding site and/or DNA barcode binding site comprising the nucleotide sequence of SEQ ID NO: 1 or the sequence shown in FIG. 6B. In certain embodiments, the poly-T end detects a VHH antibody and endogenous mRNA of the cell. In one embodiment, the two bead codes comprise SEQ ID NO: 1-2, where SEQ ID NO: 1 is the barcode capture site (described above) and SEQ ID NO: 2 is the mRNA capture site which is a repeat of 20 T residues (TTT TTT TTT TTT TTT TTT TT) (SEQ ID NO: 2).

In other embodiments, the disclosure provides for kits for characterizing a lipid nanoparticles for in vivo delivery of an agents. The kits include the beads for characterizing a lipid nanoparticles and optionally instructions for use.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein. The described embodiments and following examples are for illustrative purposes and are not intended to limit the scope of the claims. Other modifications, uses, or combinations with respect to the compositions described herein will be apparent to a person of ordinary skill in the art without departing from the spirit and scope of the claimed subject matter.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### Example 1: Design and Testing of Single-Cell Nanoparticle Targeting-Sequencing

Cells that were previously described as homogenous are composed of subsets with distinct transcriptional states. However, it remains unclear whether this cell heterogeneity influences the efficiency with which lipid nanoparticles (LNPs) deliver mRNA therapies in vivo. To test the hypothesis that cell heterogeneity influences LNP-mediated mRNA delivery, anew method of testing multiomic nanoparticle delivery system called Single-cell Nanoparticle Targeting-sequencing (SENT-seq) was devised. SENT-seq quantifies how dozens of LNPs deliver DNA barcodes and mRNA into cells, subsequent protein production, and the transcriptome, with single-cell resolution. Using SENT-seq, it is possible to identify cell subtypes that exhibit particularly high or low LNP uptake as well as genes associated with those subtypes.

Single-Cell Readouts of Gene Expression, mRNA Delivery, and DNA Barcode Delivery

Single-cell Nanoparticle Targeting-sequencing (SENT-seq) quantifies the biodistribution of many chemically distinct LNPs, measured with DNA barcodes; the functional delivery of mRNA, measured as protein using DNA-encoded antibodies; and the transcriptome of transfected cells, measured with single-cell RNA sequencing (scRNA-seq) (see FIG. 1A).

SENT-seq was initiated by formulating LNP-1, with chemical structure 1, to carry mRNA encoding a glycosylphosphatidylinositol (GPI)-anchored camelid VHH antibody (anchored-VHH, aVHH) and DNA barcode 1 at a lipid:nucleic acid mass ratio of 10:1 using microfluidic mixing³³. This process was repeated N times so that LNP-N, with chemical structure N, was formulated to carry aVHH mRNA and DNA barcode N. With DNA barcodes used to quantify biodistribution from many LNPs simultaneously, SENT-seq can test a large, chemically diverse LNP library without the need to sacrifice, sort, and sequence single cells from hundreds of mice. The aVHH, barcode, and mass ratio were rationally designed:the VHH domain was linked with a GPI anchor to induce cell-surface aVHH expression, allowing aVHH+ cells to be detected with an anti-camelid VHH antibody³⁴; the DNA barcode (FIG. 6A) was sequence optimized to reduce genomic DNA background and chemically modified to reduce nuclease-mediated degradation³⁵; and the 10:1 mass ratio has successfully delivered mRNA while retaining enough barcode to read out¹¹.

After administering the barcoded LNP library to mice, the liver was isolated and digested into a single-cell suspension which was then mixed with 20 μm carboxyl-coated magnetic polymer beads conjugated to DNA via an amine-reactive oligo using N-hydroxysulfosuccinimide sodium salt (Sulfo-NHS). The beads were designed with two orthogonal capture sequences: one bound a universal sequence in all the LNP-carried DNA barcodes, while the other, a poly-T, captured poly-A tagged cell hash oligo antibodies36 and endogenous mRNA with poly-A tails (FIG. 1, FIG. 6B). It was reasoned that by adding orthogonal capture sequences to the same bead in defined ratios, the proportion of sequencing reads—and therefore sensitivity—of LNP-delivered DNA barcodes, relative to the mRNA and protein readouts, could be customized. To evaluate whether distinct capture signals orthogonally quantified LNP barcodes and mRNA, the beads were coated with the LNP barcode capture sequence and poly-T capture sequence, mixed them with 10 μM of complementary fluorescent probes for 15 minutes, washed them, and then quantified probe mean fluorescent intensity (MFI) using flow cytometry. Beads that were mixed with fluorescent probes complementary to the barcode capture sequence or, separately, the poly-T capture sequence led to increased MFI in the appropriate channels, whereas beads mixed with both fluorescent probes resulted in a strong signal in both channels (FIG. 1C). As a negative control, MFI was quantified after mixing both probes with beads without capture sequences and found no signal (FIG. 1C). A titration experiment was then performed, decreasing the amount of barcode while increasing the amount of mRNA, or vice versa (FIG. 1D). The relationship between barcode and mRNA concentrations and subsequent mapped reads was linear across five orders of magnitude (FIG. 1E).

SENT-seq utilizes orthogonal capture sequences to generate tunable multiomic readouts (FIG. 1A-E). After formulating and injecting N chemically distinct LNPs to carry mRNA and DNA barcodes, tissues were isolated and digested into single-cell suspensions. Delivery mediated by all N LNPs, subsequent mRNA-mediated protein production, and transcriptome was quantified in single cells using next-generation sequencing (FIG. 1A). The sensitivity of the DNA barcode readouts relative to the biological (i.e., mRNA and protein) readouts was controlled by the ratio of two orthogonal capture sequences:the barcode capture sequence and the poly-T, which captured mRNA and poly-A tagged cell hash oligo antibodies (FIG. 1B). Mean fluorescent intensity (MFI) after beads carrying the barcode capture sequences and poly-T were mixed with the fluorescent complementary barcode probe, fluorescent poly-A probe, both, or as a negative is shown in FIG. 1B. FIGS. 1D and E show read standard curves after beads carrying both capture sequences were mixed with varying amounts of LNP barcodes or mRNA. Barcode (BC), linker (L), unique molecular identifier (UMI).

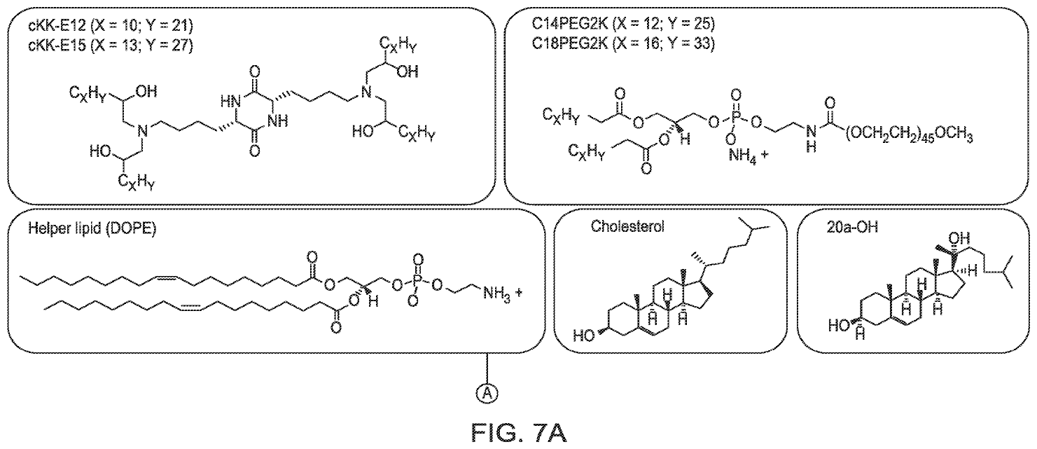

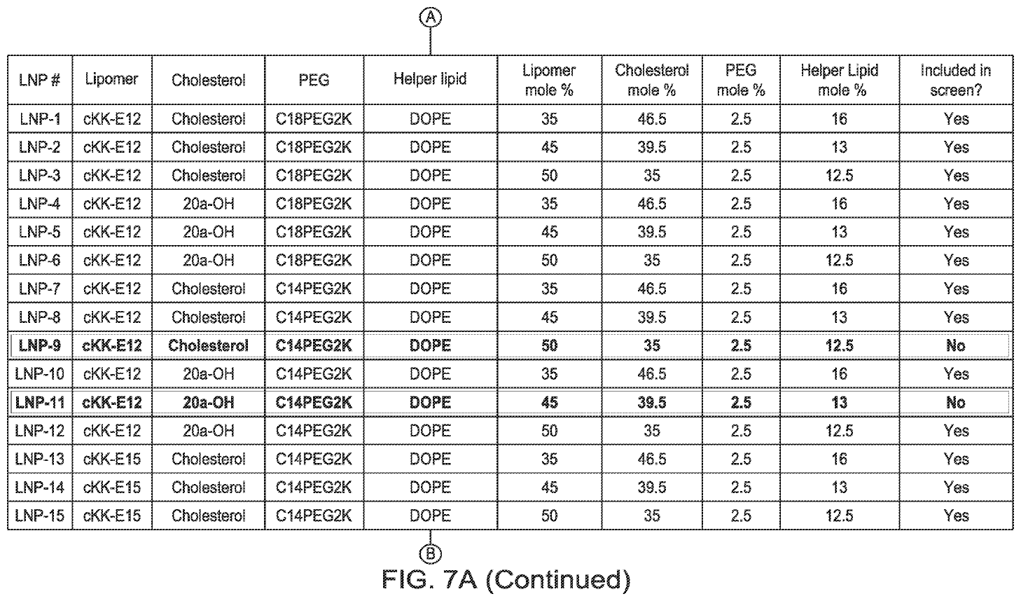

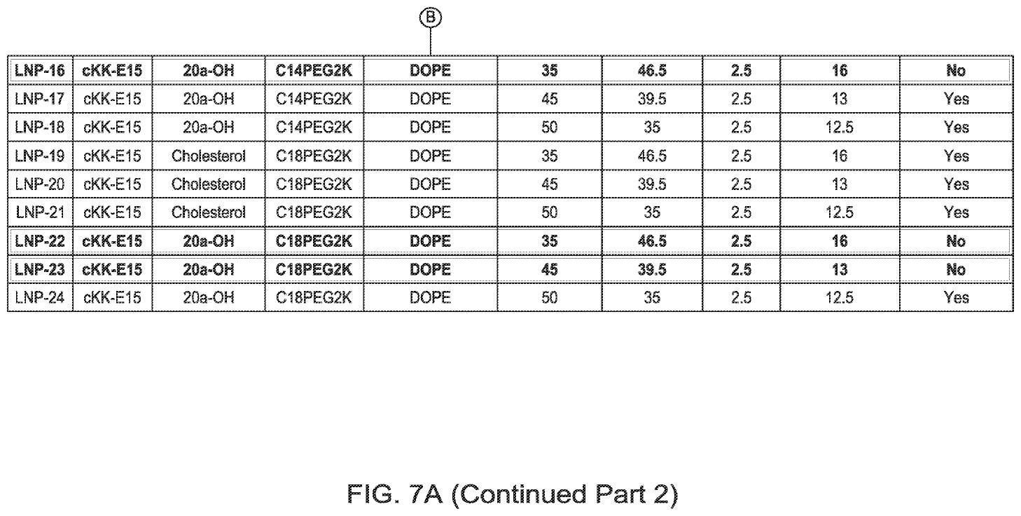

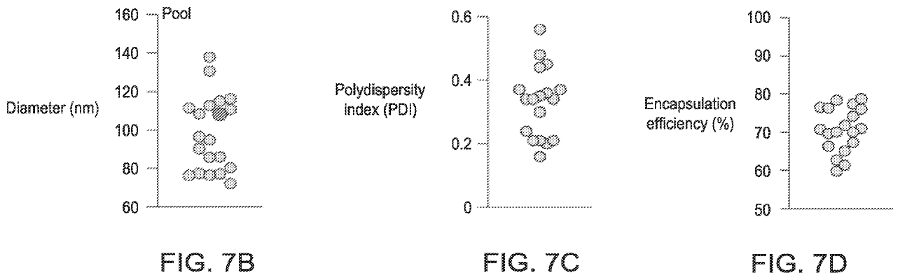

SENT-seq was then used to analyze the presence of LNP-delivered DNA barcodes, functional LNP-mediated mRNA delivery, and the transcriptome, using 24 chemically distinct LNPs in vivo. To create the 24 LNPs (FIG. 7A), the four traits that can alter LNP activity were varied³⁷: the identity of three of the constituents (ionizable lipid, cholesterol, or PEG-lipid) and the molar ratio of all four constituents. The hydrodynamic diameter and stability of all 24 LNPs using dynamic light scattering (DLS) were then characterised. LNPs with a unimodal diameter distribution and a hydrodynamic diameter between 50 and 150 nm (FIG. 7B,C) were pooled and dialyzed in 1×PBS. Additionally, the encapsulation efficiency of all pooled LNPs individually and found that they were all over 60% (FIG. 7D) was measured. As a control, hydrodynamic diameter of the pooled LNPs was measured and found to be within the range of diameters of the LNPs constituting the pool, suggesting that LNPs did not aggregate after mixing (FIG. 7B). Of the 24 LNPs, the 19 that met these inclusion criteria were administered as a pool to mice at a total nucleic acid dose of 1.5 mg/kg (0.08 mg/kg/LNP, on average). As a negative sequencing control, unencapsulated barcode (also termed naked barcodes), which enter cells far less efficiently than barcodes encapsulated by LNPs, were added⁹.

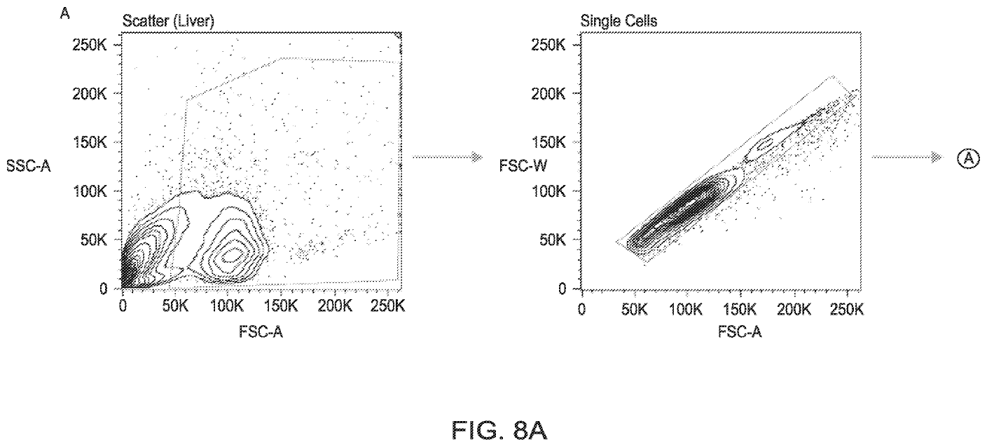

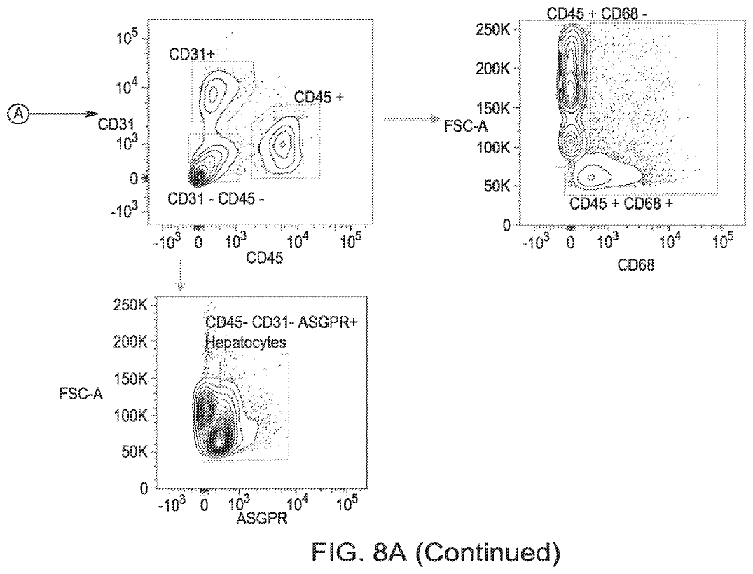

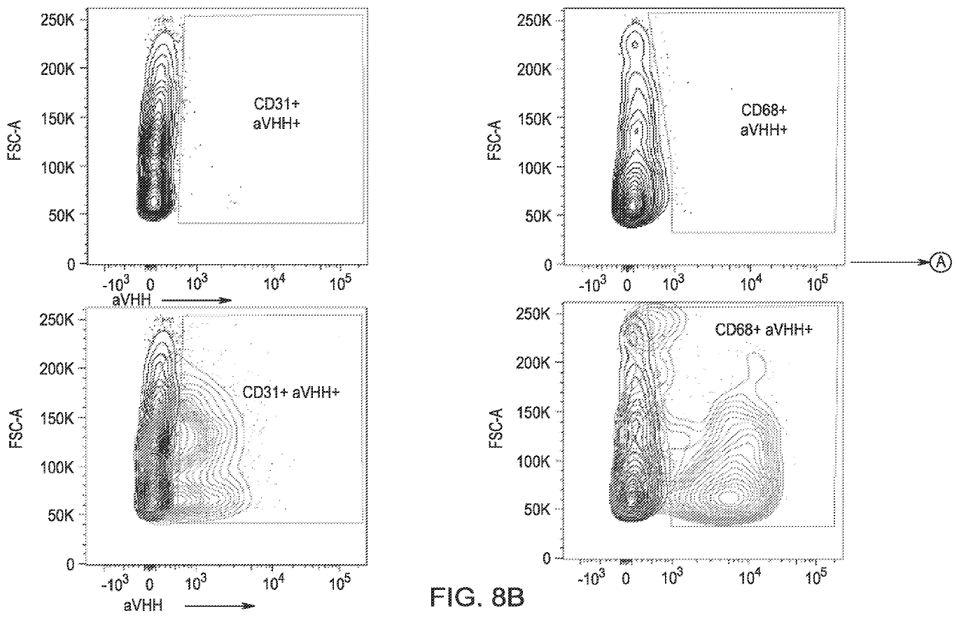

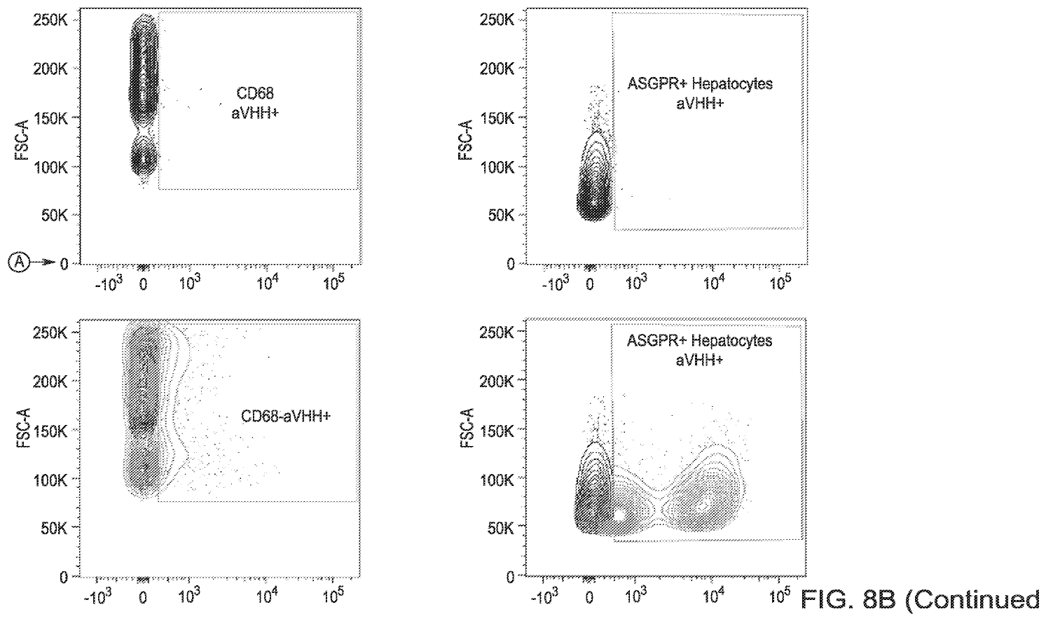

Fifteen hours after administration, which is sufficient time for LNP-mediated aVHH mRNA delivery to produce aVHH protein, cells were isolated from the liver, digested into a single-cell suspension, and live cells were sorted using FACS (FIG. 8). A Microwell-seq protocol³⁸ was modified to read out both mRNA and barcode at the single-cell level. Then the scRNA-seq data was first analyzed using Seurat and plotted data from 12,828 distinct single cells using t-distributed stochastic neighbor embedding (t-SNE). The number of cells per condition, reads per cell, genes per cell, total reads, and a break-down of the percentage of reads mapped to cellular mRNA were consistent with previous publications³⁸ and are shown in Table 1-1 below.

TABLE 1-1
Table showing single-cell RNA-seq data

| | Mean | Median | Mean | Median | Mean | Median | | | % | % |
|---|---|---|---|---|---|---|---|---|---|---|
| | reads per | reads per | gene | gene | UMI | UMI | Number | Total | mapped | mapped |
| | cell | cell | count | count | count | count | of Cells | Counts | to Exon | to Intron |
| 1_PBS | 11751 | 2823 | 2175 | 1920 | 2320 | 2078 | 1499 | 5272051 | 66.20% | 33.80% |
| 2_PBS | 11216 | 2765 | 2277 | 2005 | 2422 | 2150 | 540 | 2020180 | 75.05% | 24.95% |
| 3_PBS | 10044 | 2640 | 2215 | 1870 | 2359 | 2030 | 622 | 1831709 | 63.70% | 36.30% |
| 4_PBS | 9694 | 2695 | 2414 | 2168 | 2558 | 2335 | 1532 | 4527792 | 75.06% | 24.94% |
| 1_Pool | 10191 | 2700 | 2272 | 2010 | 2425 | 2160 | 3330 | 10091943 | 69.97% | 30.03% |
| 2_Pool | 13006 | 2930 | 2254 | 1980 | 2408 | 2125 | 1796 | 7614914 | 67.85% | 32.15% |
| 3_Pool | 9929 | 2713 | 2199 | 1955 | 2366 | 2113 | 1706 | 4960815 | 66.23% | 33.77% |
| 4_Pool | 9371 | 2790 | 2379 | 2035 | 2701 | 2180 | 1803 | 5055979 | 67.15% | 32.85% |
| Mean | 10650 | 2757 | 2273 | 1993 | 2445 | 2146 | 1604 | 5171923 | 68.90% | 31.10% |
| Standard | 1158 | 85 | 79 | 83 | 117 | 84 | 805 | 2543331 | 3.92% | 3.92% |
| Deviation | | | | | | | | | | |

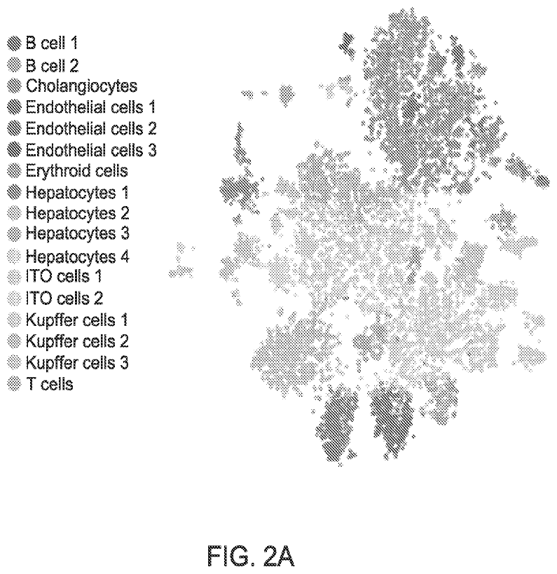

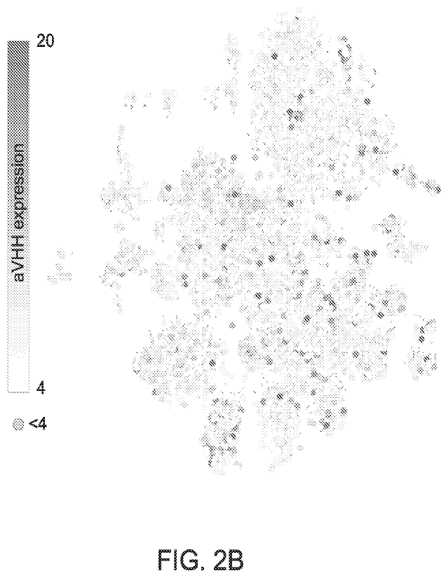

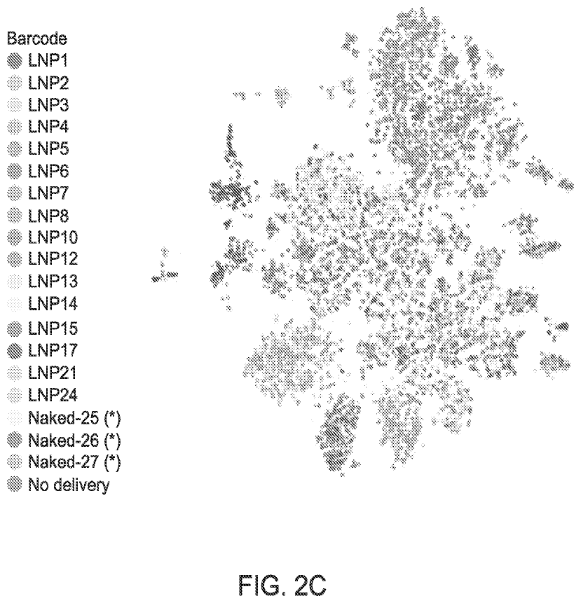

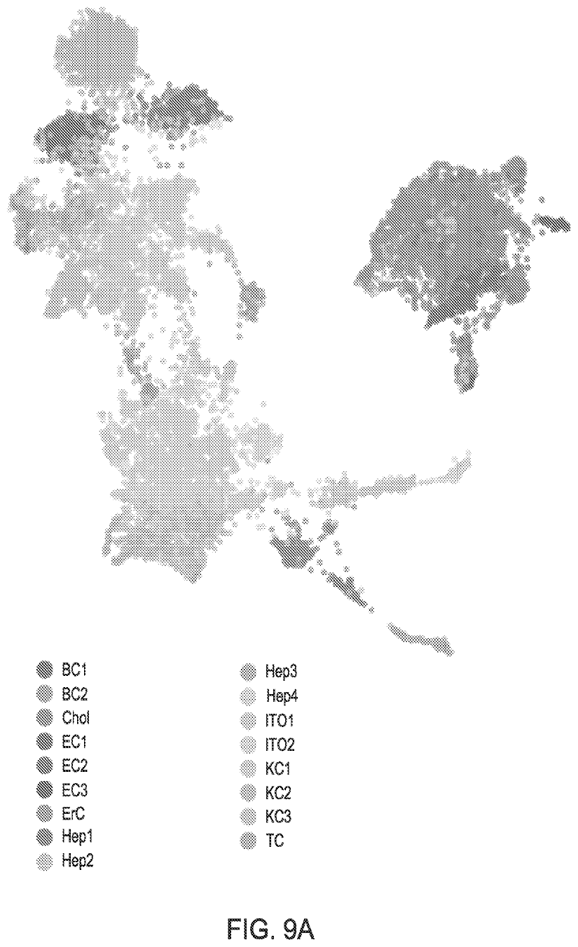

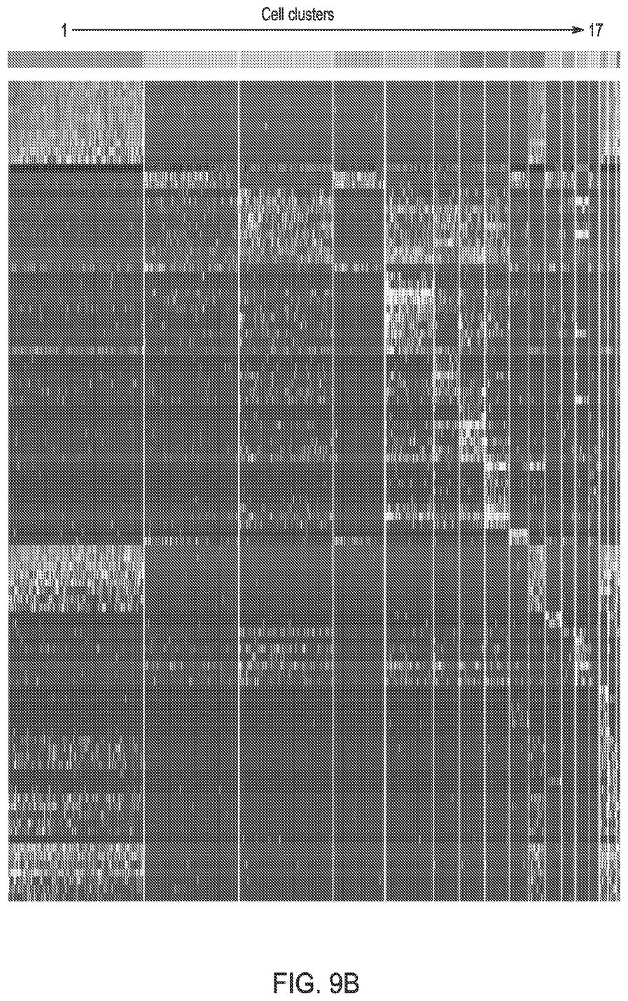

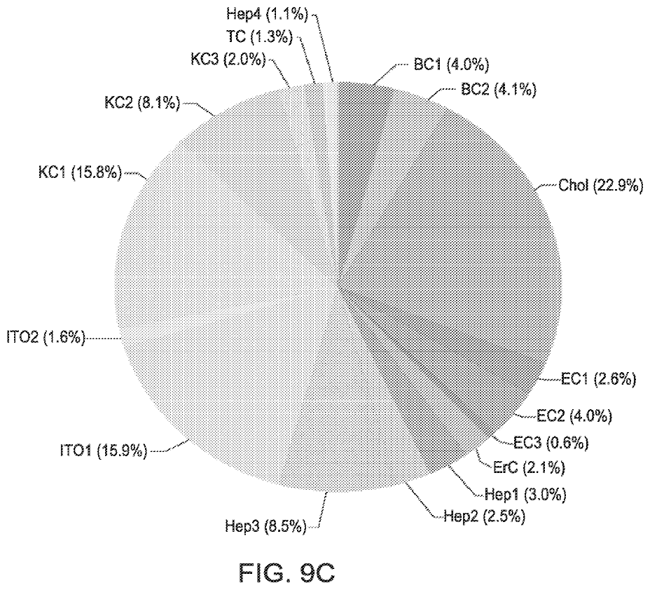

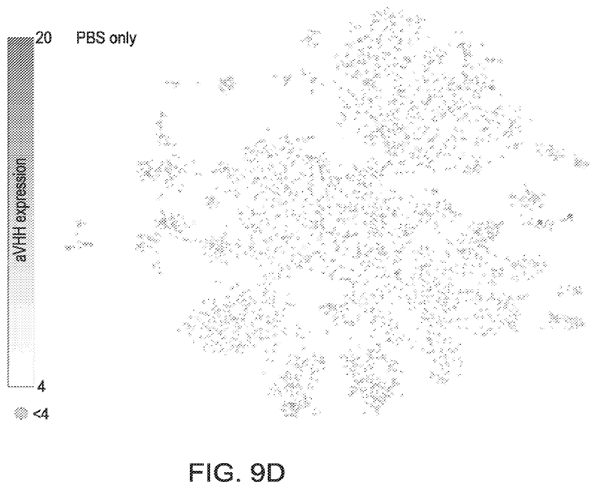

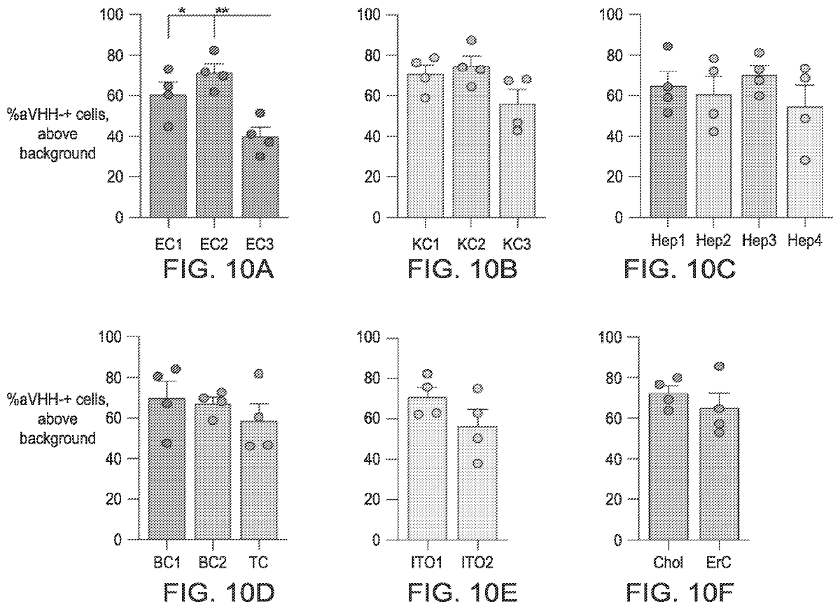

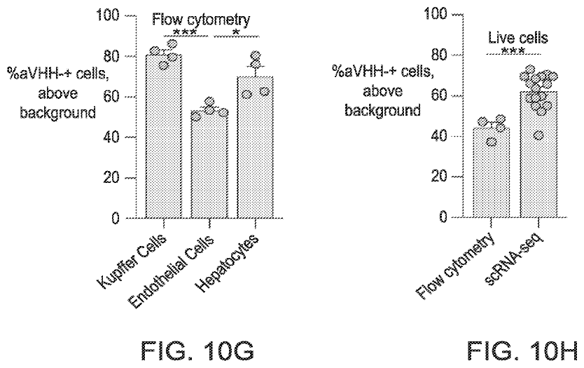

It was observed that hepatocytes, endothelial cells, Kupffer cells, hepatic stellate (Ito) cells, and other hepatic cell types separated into transcriptionally distinct subtypes when plotted using t-SNE (FIG. 2A) and UMAP (FIG. 91B), based on differentially expressed genes (FIG. 9B,C). The functional mRNA delivery (i.e., the presence of aVHH protein) at the single-cell level was then quantified by sequencing DNA-tagged anti-aVHH antibodies and overlaid these readouts with the t-SNE plot. As a control, the validity of the aVHH cutoff (>=4 reads per cell) was assessed by quantifying aVHH+ cells in control mice treated with 1×PBS and found that 10.9% of cells passed this threshold (PBS mean aVHH reads per cell: 0.5, PBS median aVHH reads per cell: 0, LNP pool mean aVHH reads per cell: 5.4, LNP pool median aVHH reads per cell: 5), indicating that our cutoff was stringent (FIG. 9D). As another control, the percentage of aVHH+ cells measured by the DNA-tagged anti-aVHH antibodies (FIG. 10A-G) was compared to the percentage of aVHH+ cells identified using traditional flow cytometry (FIG. TOG). 1.4-fold more aVHH+ cells using the DNA-tagged anti-aVHH antibodies compared to flow cytometry were found when looking at the whole hepatic population (FIG. 10H), suggesting that DNA-tagged antibodies may provide a more sensitive readout of functional mRNA delivery than flow cytometry.

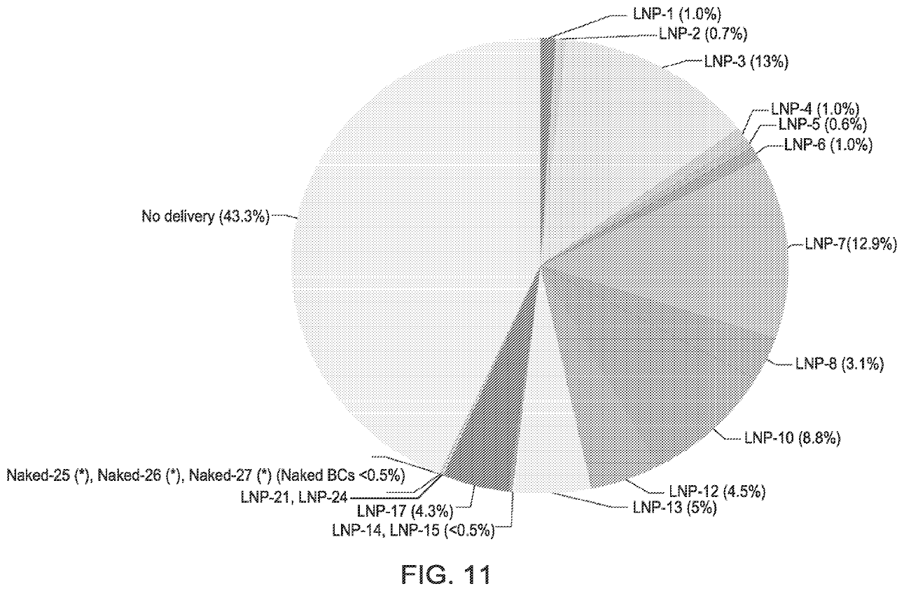

aVHH protein was observed in all 17 cell subtypes (FIG. 2B), including subtypes that are not identifiable using established FACS markers; these data demonstrate that measuring delivery in transcriptionally defined cells may generate a more detailed picture of on- and off-target delivery than traditional techniques. Finally, LNP barcode delivery in single cells was quantified (FIG. 2C) and overlaid the most common barcode in every cell on the t-SNE plot. As a control, the percentage of barcode-containing cells in control mice treated with 1×PBS was quantified and found that only 4.9% of cells passed this threshold (PBS mean barcode expression: 12.2, PBS median barcode expression: 0; LNP pool mean barcode expression: 413.2, LNP pool median barcode expression: 393.5). It was also noted that barcodes delivered by LNPs 3, 7, 10, and 12 were delivered in more cells than barcodes delivered by other LNPs (FIG. 2C) and that as expected, the negative control unencapsulated barcodes were delivered less efficiently than barcodes carried by LNPs (FIG. 11). Taking these findings together, it was concluded that it was feasible to quantify gene expression, the presence of LNP-delivered barcodes delivered by chemically distinct LNPs, and functional mRNA delivery with single-cell resolution in vivo.

### Cell Heterogeneity Influences LNP Delivery In Vivo

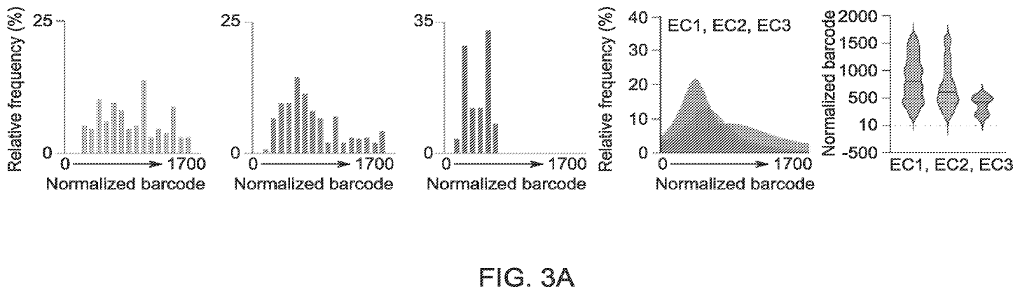

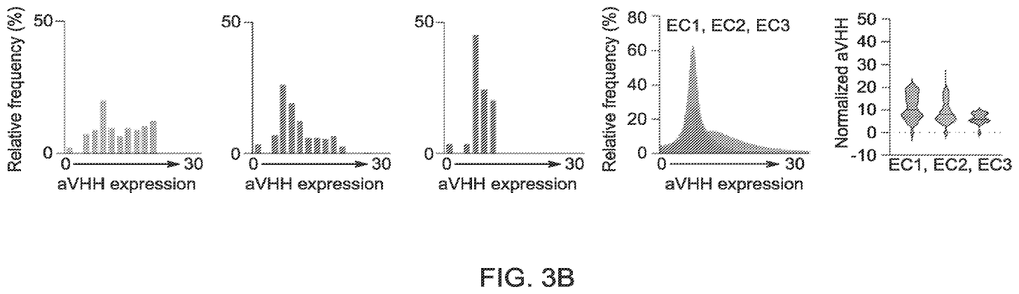

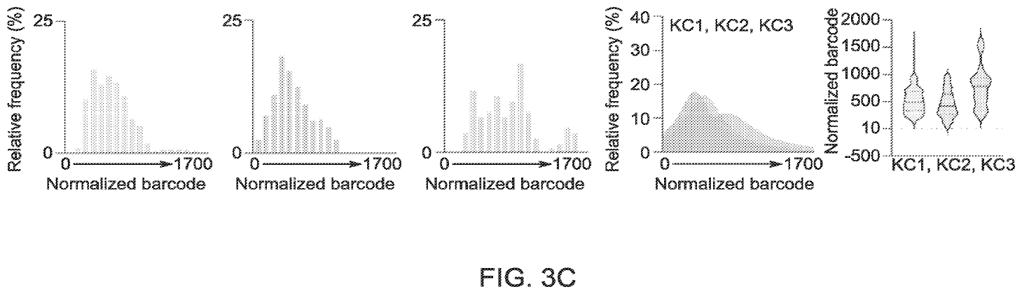

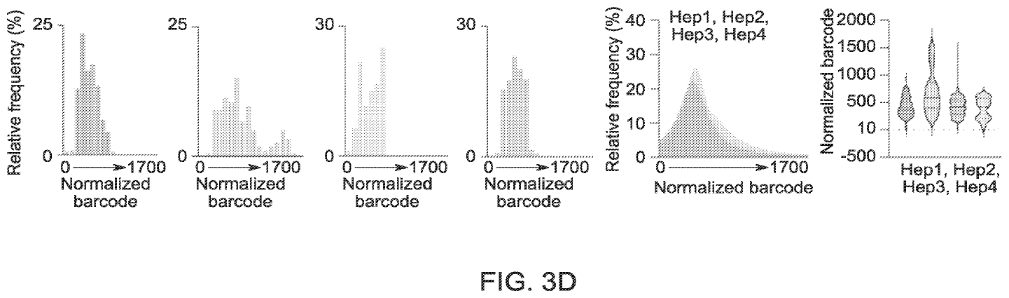

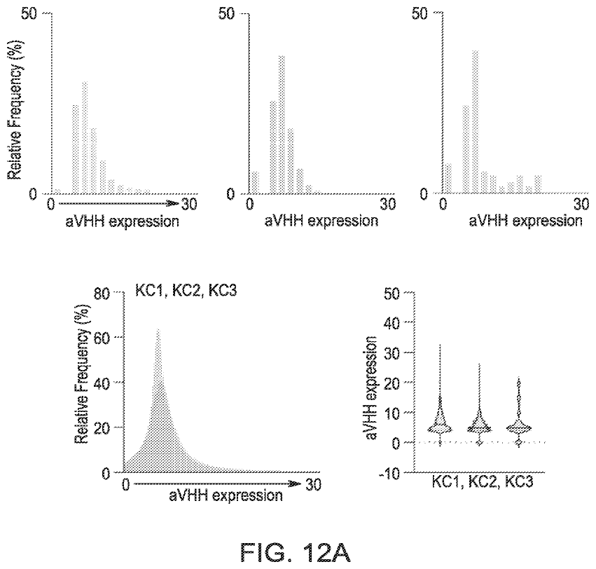

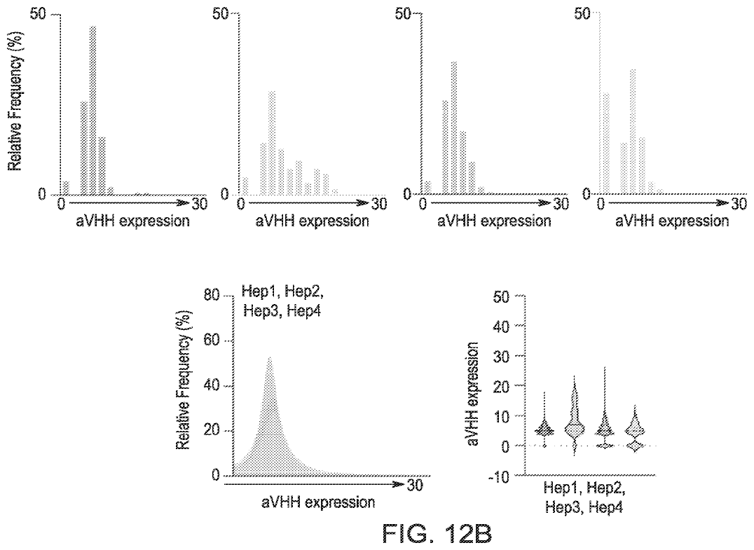

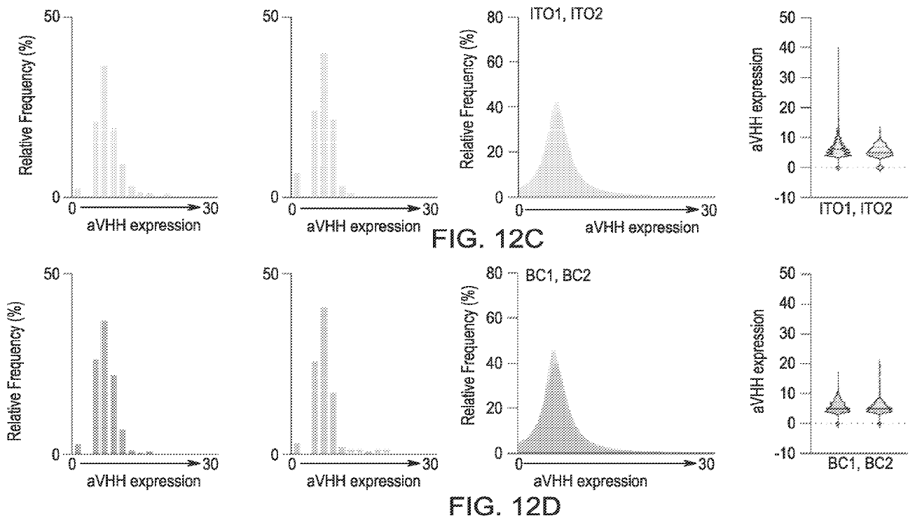

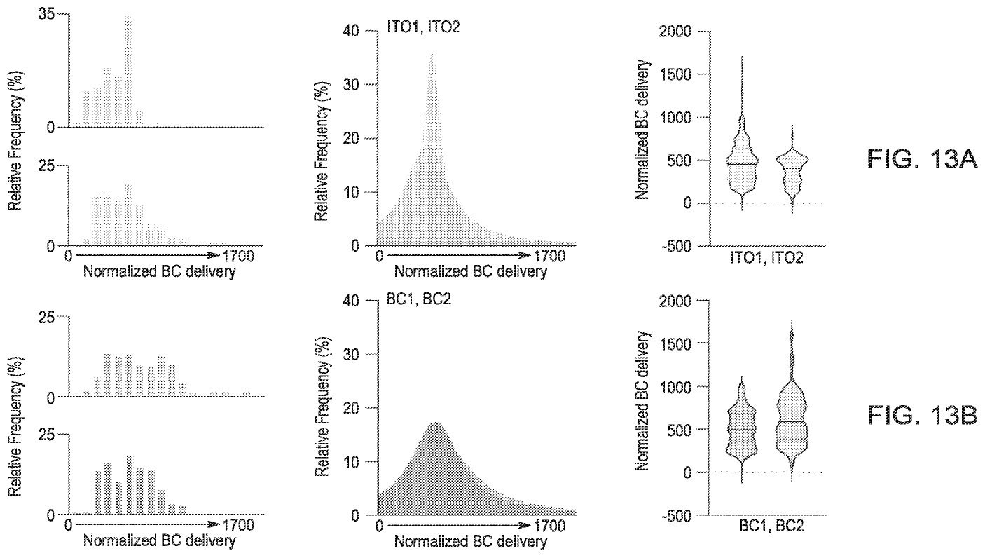

After characterizing SENT-seq and using it to generate multiomic nanoparticle readouts, the data was used to test the hypothesis that cell heterogeneity influences LNP delivery. LNP-mediated DNA barcode delivery was quantified by quantifying the barcode counts in each cell, binning those counts by increments of 100, and plotting a histogram of cells with counts within each bin. Notably, different cell subtypes exhibited distinct levels of barcode reads. For example, endothelial cell subtype three (EC3) had a sharp peak (mean: 367 counts, median: 420 counts), whereas endothelial cell subtype one (EC1) had a broader peak (mean: 845 counts, median: 799 counts) but included cells generating as few as 100 counts and cells generating as many as 1,700 counts (FIG. 3A). To complement these DNA barcode readouts of LNP biodistribution, aVHH protein reads, which occur when LNP-delivered aVHH mRNA is translated into functional aVHH protein, were analyzed. aVHH counts were binned by increments of 2, the percentage of cells with aVHH expression values within each bin was plotted, and it was found that the aVHH profiles for endothelial cells were similar to LNP barcode delivery profiles (FIG. 3B). A similar qualitative trends in Kupffer cell subtype three (KC3) relative to Kupffer cell subtype one (KCl), and Kupffer cell subtype two (KC2) (FIG. 3C, FIG. 12A), hepatocyte subtype two (Hep2) compared to hepatocyte subtypes one (Hep1), three (Hep3), and four (Hep4) (FIG. 3D, FIG. 12B) was noted. By contrast, Ito subtype one (ITO1) and two (ITO2) (FIG. 12C, FIG. 13A) had similar aVHH expression profiles but different barcode expression profiles, while B cell subtypes one (BC1) and two (BC2) (FIG. 12D, FIG. 13B) had similar aVHH and normalized barcode expression profiles.

Transcriptional Analysis of Cells that Exhibit Differential LNP Delivery

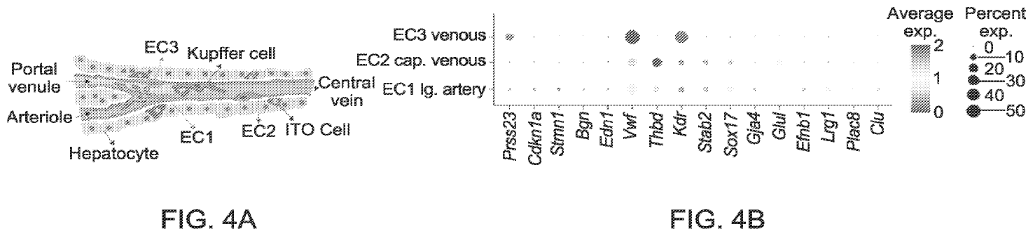

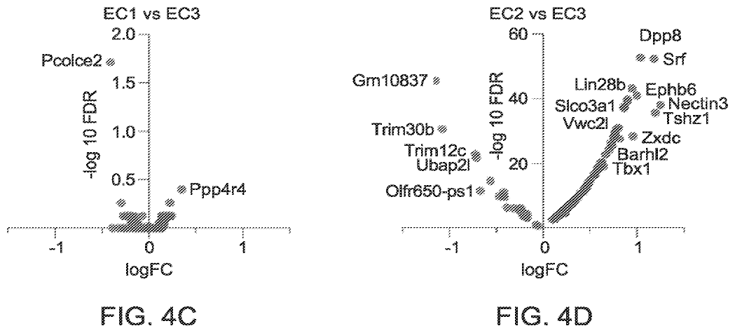

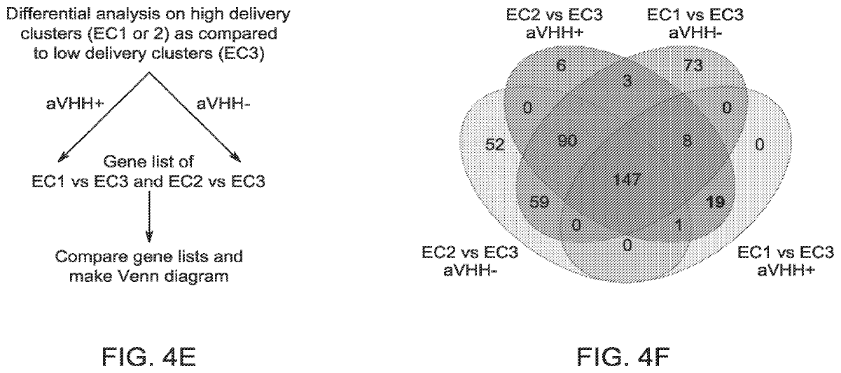

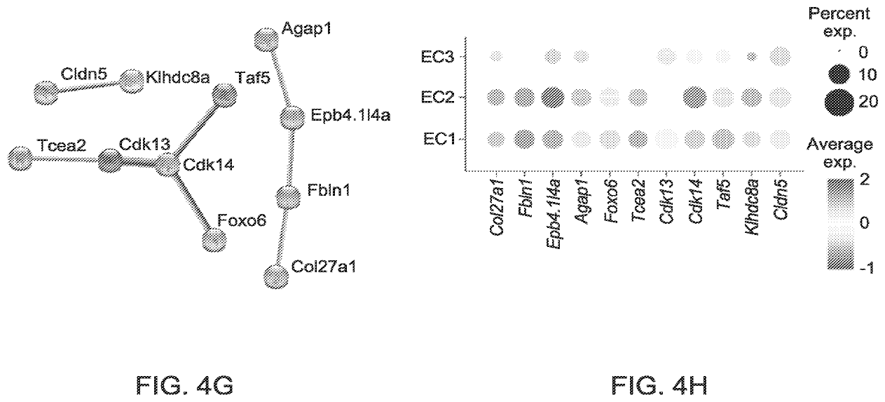

These data led to focus on endothelial cells, which had the most distinct subtype-dependent LNP delivery and the largest statistically significant differences in the percentage of aVHH+ cells (FIG. 10A). Notably, RNA sequencing can determine the positioning of a given endothelial cell within the vascular tree39 (i.e., artery, capillary, vein, FIG. 4A). Therefore 20 genes previously reported to determine endothelial location in the liver vascular tree³⁹ were evaluated and it was found that 16 were expressed at sufficiently high levels to analyze. Genes Vwf and Thbd were highly expressed in EC1, Thbd in EC2, and Kdr and Prss23 in EC3. These data were consistent with previous work³⁹ and suggested that EC1 was part of a large artery, EC2 was part of the capillary venous system, and EC3 was part of the general venous system (FIG. 4B). To better understand global differences in gene expression profiles, the analysis was expanded to include all genes with statistically significant differences in expression between EC1 and EC3 and, separately, EC2 and EC3 (FIG. 4C, D). The DAVID database, which identifies pathways associated with a list of genes, found that genes upregulated in EC3, relative to EC1, were either associated with transcription factor binding (P value=6.8×10−3, GO:0008134) or with DNA binding (P value=4.3×10−3, GO:0003677). This further suggests that the cell types were transcriptionally distinct.

Although these analyses revealed subtypes that were transcriptionally distinct, they could not identify genes that may drive differences in LNP delivery. For example, if all cells in EC1 are compared to all cells in EC3, including cells that were not targeted by LNPs, the data include differences in basal gene expression that are unrelated to LNP delivery; this basal gene expression problem limits all RNA sequencing-based analyses of nanoparticle delivery. SENT-seq was specifically engineered to alleviate this issue by enabling us to perform three key analytical steps (FIG. 4E). First, only cells in EC3 and EC1 or EC2 that had functional aVHH delivery (aVHH counts>4, denoted as aVHH+) were compared. Second, separately aVHH− (aVHH counts<4, denoted as aVHH−) cells in EC3 to aVHH− EC1 or EC2 were compared, thereby generating a list of genes that were differentially expressed without functional LNP delivery, i.e., “background” genes. Third, the background genes were removed from the list of differentially expressed genes in our aVHH+EC3 and aVHH+EC1 or EC2 comparisons. Using this approach, 19 differentially expressed genes in aVHH+EC1 and EC2, relative to EC3, that were not differentially expressed in aVHH− cells (FIG. 4F bolded) were identified. After inputting these 19 genes into the Search Tool for the Retrieval of Interacting Genes/Proteins (STRING)⁴⁰, it was observed that 11 of these genes had significant interactions with each other (FIG. 4G, Table 2).

Table 1-2 shows the 11 differentially regulated genes in aVHH+EC1 and EC2, relative to EC3, that were not differentially expressed in aVHH− cells, and the current putative roles for those genes in Mus musculus.

TABLE 1-2
Differentially regulated genes in aVHH+ EC1 and EC2, relative to
EC3, that were not differentially expressed in aVHH− cells

| Gene | Putative Role |
|---|---|
| Col27a1 | Preproprotein linked to playing a crucial roles in tissue growth and |
| | repair |
| Fbln1 | May play a role in cell adhesion and migration along protein fibers |
| | within the extracellular matrix |
| Ebp4.1I4a | Thought to play an important role in regulating interactions between |
| | the cytoskeleton and plasma membrane, while also being involved in |
| | the beta-catenin signaling pathway |
| Agap1 | Directly and specifically regulates the adapter protein 3-dependent |
| | trafficking of proteins in the endosomal-lysosomal system |
| Foxo6 | Transcriptional activator |
| Tcea2 | Necessary for efficient RNA polymerase II transcription elongation |
| Cdk13 | Cyclin-dependent kinase which displays CTD kinase activity and is |
| | required for RNA splicing |
| Cdk14 | Kinase involved in the control of the eukaryotic cell cycle, including |
| | regulating the Wnt signaling pathway, cell cycle progression and cell |
| | proliferation. |
| Taf5 | A component of the transcription factor IID complex, which is |
| | essential for mediating regulation of RNA polymerase transcription |
| Klhdc8a | Putatively linked to tumors potentially maintaining aggressiveness in |
| | the absence of epidermal growth factor receptor dependence |
| Cldn5 | Plays a major role in tight junction-specific obliteration of the |
| | intercellular space, through calcium-independent cell-adhesion activity |

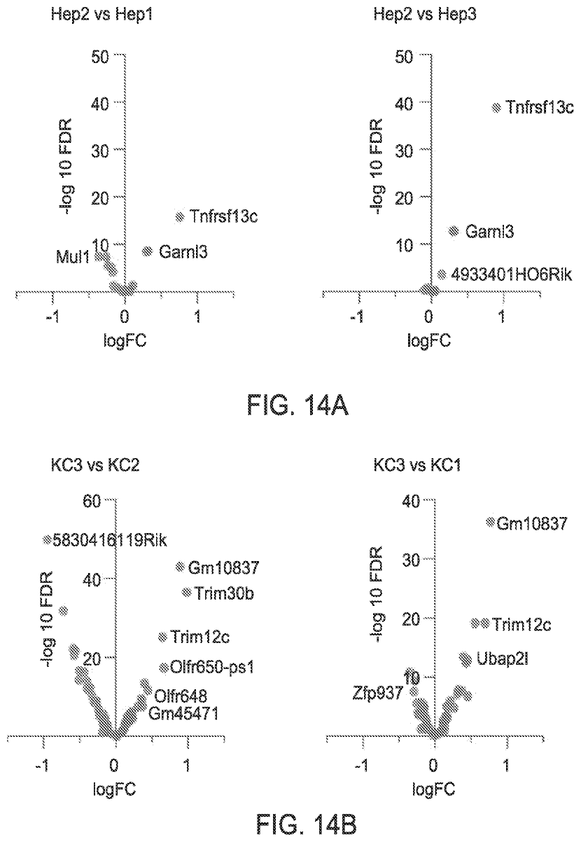

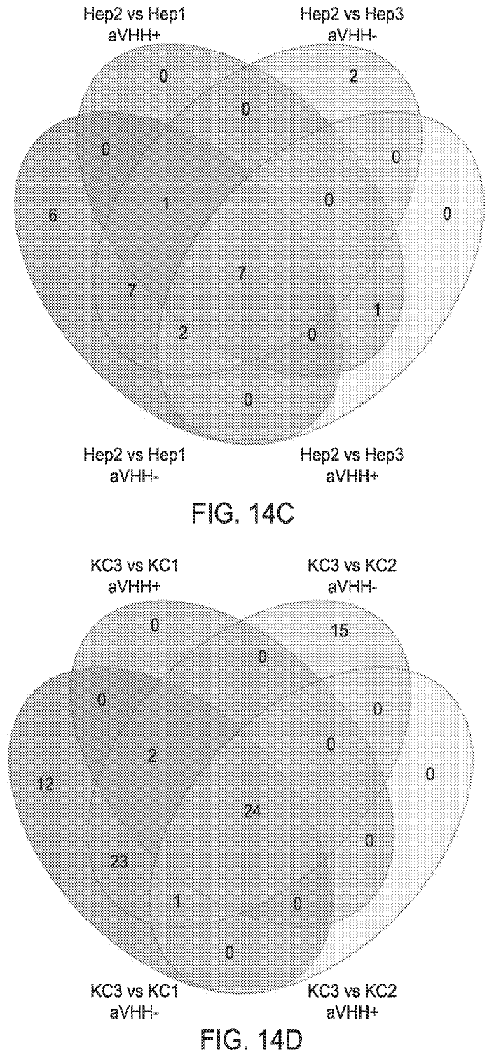

Of these genes, the nodal molecules were CDKT13 and CDK14, which are part of the cyclin-dependent kinase family⁴¹. This family of molecules has been shown to be important in regulating cell cycle and mRNA processing⁴², which may explain the increased level of functional delivery in these endothelial cell clusters. To confirm that these genes were in fact expressed differently, the overall expression levels within each cluster was compared using a dot map. It was found that the expression levels were much higher in EC1 and EC2 and much lower or even downregulated in EC3 (FIG. 4H). As noted, EC3 also had the lowest delivery profile, suggesting that downregulation of these genes may play a role in LNP-mediated mRNA delivery. These analyses were then repeated for hepatocytes (FIG. 14A, C) and Kupffer cells (FIG. 14B,D) and significantly fewer genes differentially expressed in the aVHH+ cells that were not differentially expressed in the aVHH− cells were found.

Quantifying LNP Tropism with Single-Cell Resolution

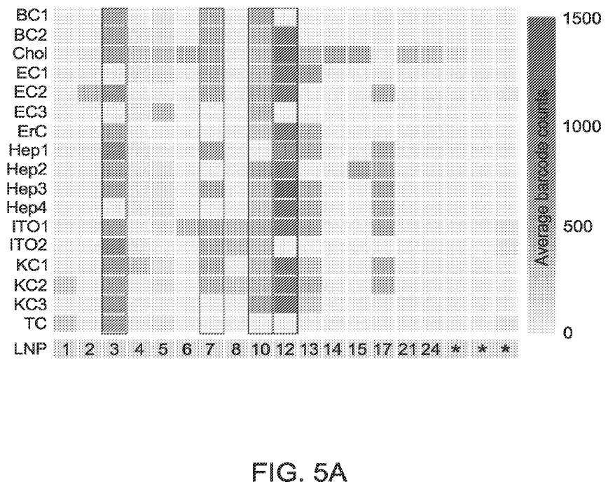

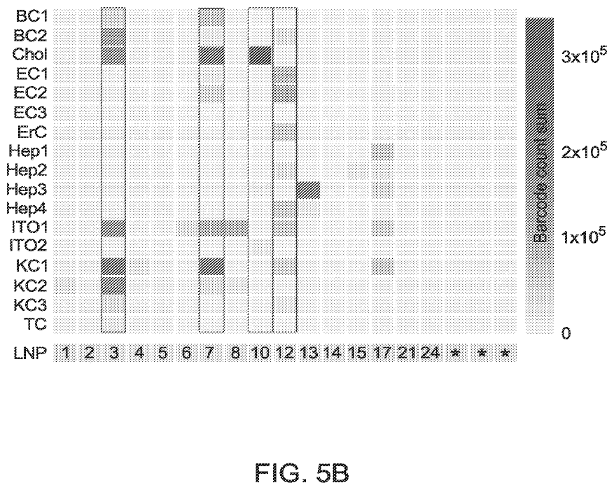

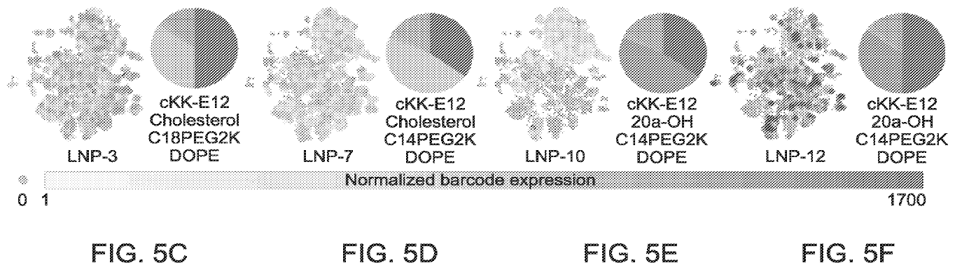

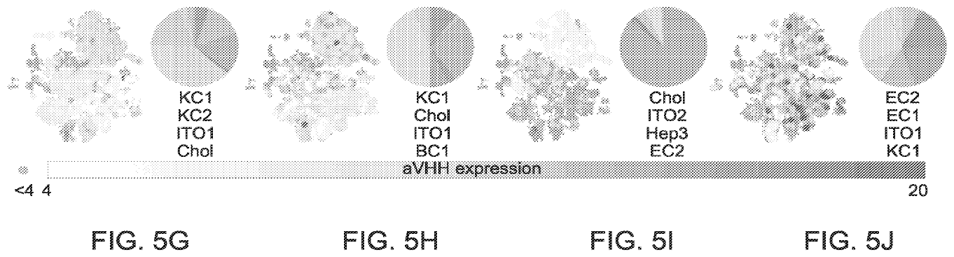

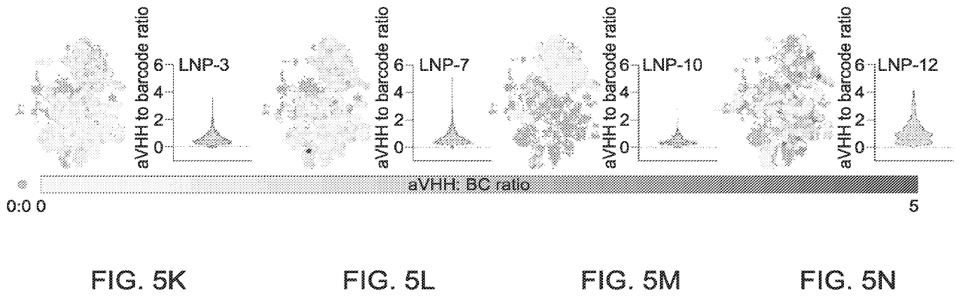

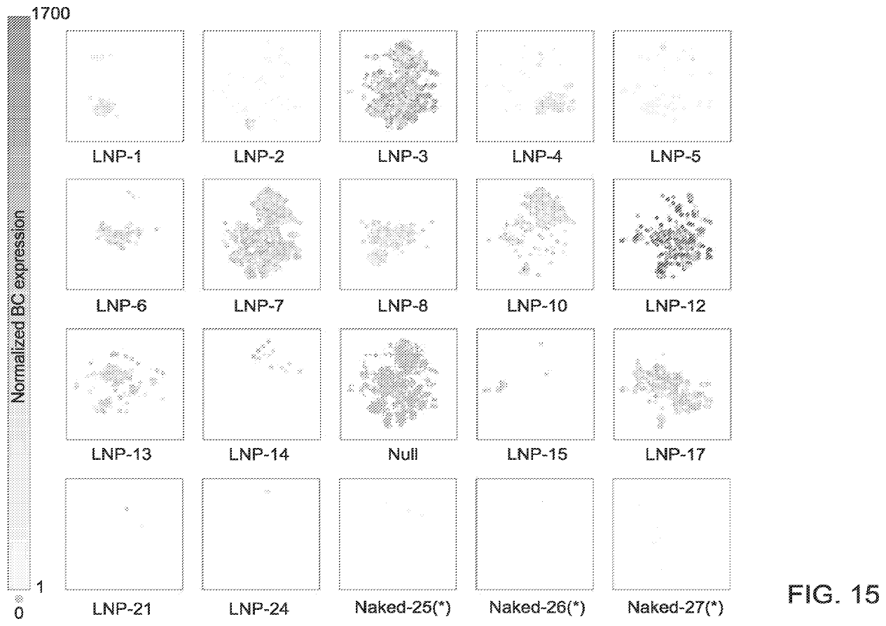

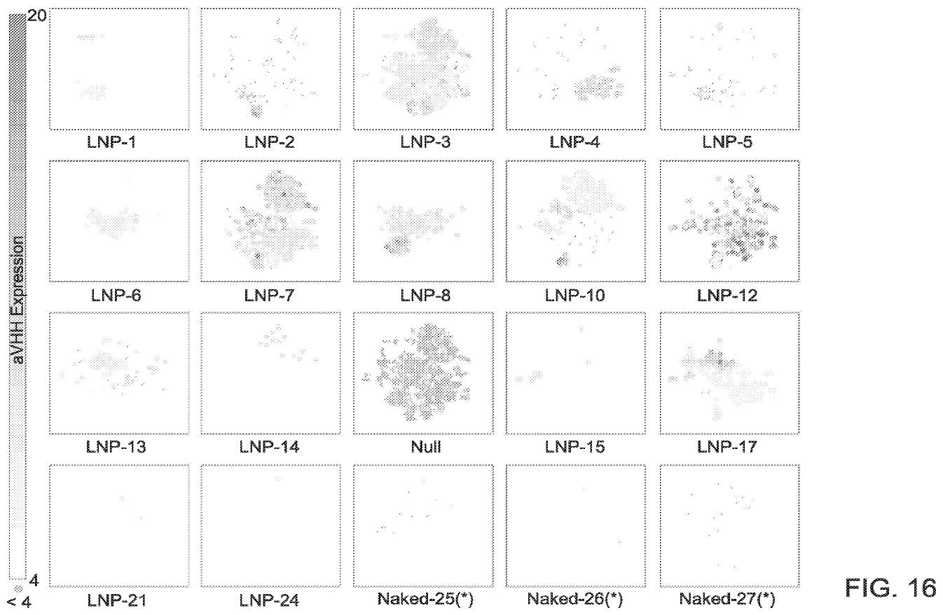

These data demonstrate that cell subsets differentially interact with LNPs, which led us to hypothesize that chemically distinct LNPs could exhibit different tropisms. Therefore the normalized barcode counts for all 17 cell subtypes as both an average (FIG. 5A) and sum (FIG. 5B) were plotted. As a control, the unencapsulated barcodes (marked by “*”) were analyzed and it was found that they were delivered less efficiently than barcodes encapsulated in LNPs. Consistent with the original overlay of the most represented barcodes on the t-SNE plot (FIG. 2C), LNPs 3, 7, 10, and 12 were overrepresented, relative to other LNPs. Then (i) the normalized barcode counts for each individual LNP (FIG. 5C-F, FIG. 15) and (ii) the aVHH expression for each individual LNP (FIG. 5G-J, FIG. 16) were plotted and this information was overlaid on the t-SNE plot. LNP-3 was enriched in KCl and KC2, followed by ITO1 and cholangiocytes (FIG. 5G).

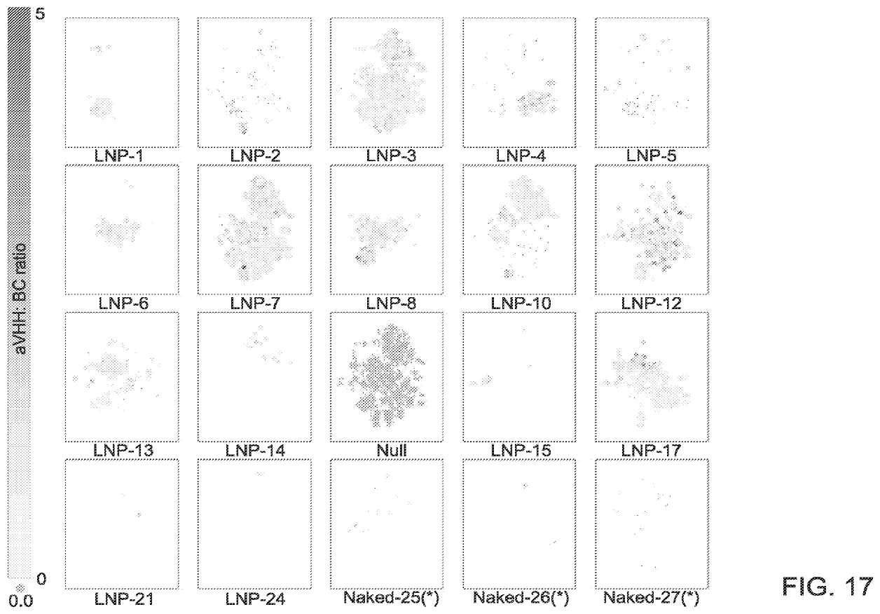

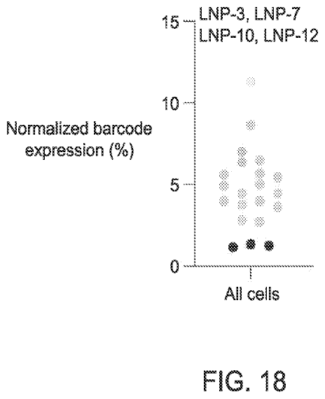

LNP-7 was enriched in KCl, cholangiocytes, ITO1, and BC1 (FIG. 5H). LNP-10 demonstrated strong tropism for cholangiocytes (FIG. 5I), and LNP-12 was enriched mostly in EC1 and EC2 (FIG. 5J). It was reasoned that these LNPs could deliver functional mRNA with different efficiencies relative to their biodistribution. This rationale is supported by evidence that LNP endosomal escape is inefficient^{43, 44} and thus LNP biodistribution readouts can differ from functional mRNA delivery readouts⁴³. To quantify this, he ratio of aVHH protein to LNP barcode in individual cells, for each LNP (FIG. 5K-N, FIG. 17), was plotted. It was found that LNP-12 tended to have a higher ratio of aVHH protein to barcode per cell, suggesting that the LNP, or the cell types it was transfecting, led to more functional delivery per unit of nucleic acid entering the cell. Taken together, these data lead to the conclusion that LNPs can have differential tropism and activity within the liver microenvironment. As a control, these single-cell readouts were compared to established bulk DNA barcoding assays45 by measuring barcodes in aVHH+ endothelial cells (CD45-CD31+), Kupffer cells (CD45+CD68+), and hepatocytes (CD31-CD45-ASGPR+) isolated by FACS (FIG. 8). Consistent with the single-cell readouts, LNPs 3, 7, 10, and 12 had the highest normalized barcode delivery (FIG. 18).

### Materials and Methods

### Synthesis of CKK-E15.

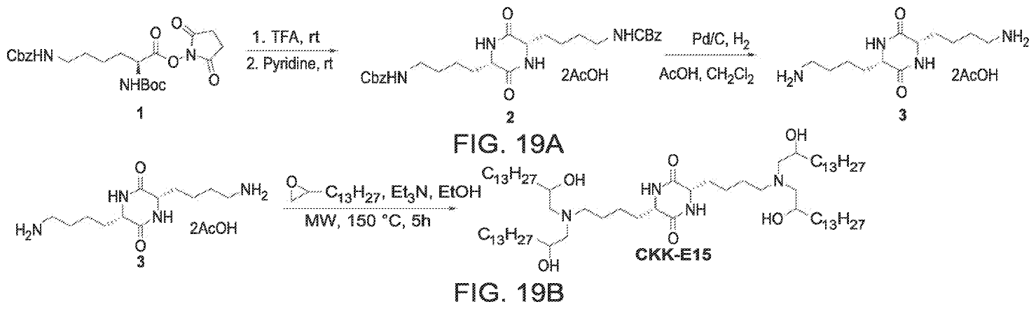

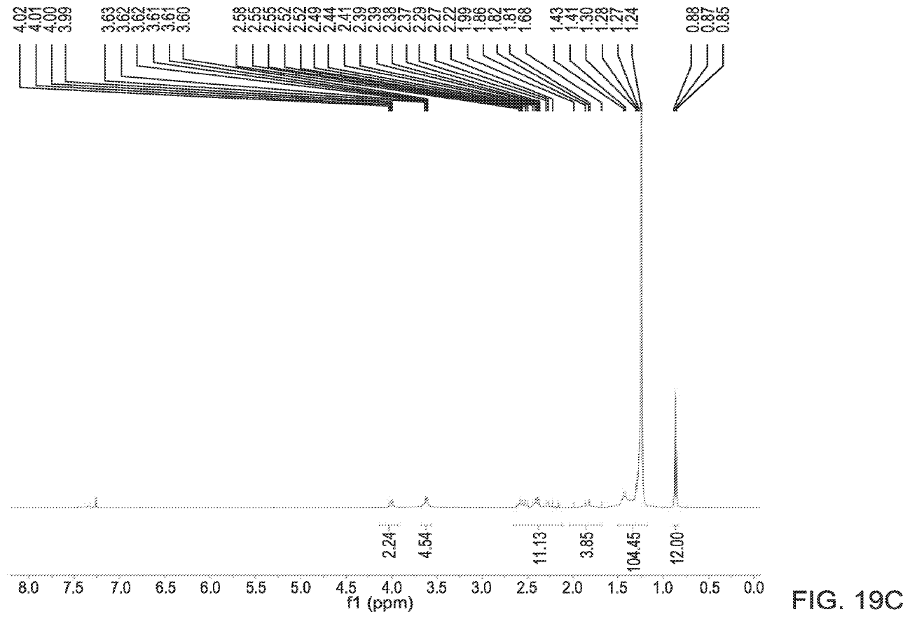

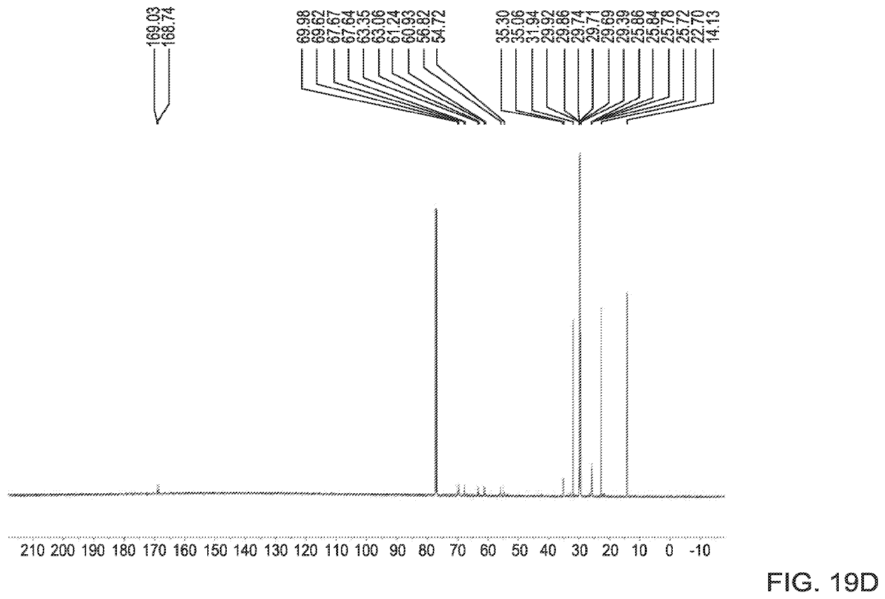

cKK-E15 was prepared as previously described 26 (FIG. 19A-D). Briefly, compound 1 (20 g, 41.9 mmol) was charged in a 100 ml flask; trifluoroacetic acid (42 mL) was added slowly at 0° C. and then stirred at room temperature for 30 min. The solvent was evaporated under reduced pressure, and then the crude product, dissolved in DMF (5 mL), was added dropwise to pyridine (300 ml) at 0° C. The reaction mixture was stirred at room temperature overnight. The solvents were evaporated under reduced pressure and the crude product washed with ethyl acetate to give pure compound 2 (8.4 g, 31% yield). To a solution of compound 2 in acetic acid/CH₂Cl₂ (1/1, 300 ml) was added Pd/C (10 wt. %, 3.0 g). The black suspension was degassed for 5 min with hydrogen and stirred at room temperature overnight under hydrogen atmosphere. The reaction mixture was filtered on Celite and washed with MeOH. The combined filtrates were concentrated, and the crude compound was washed with ethyl acetate to yield compound 3 (4.8 g, 98% yield) (FIG. 19A). To a solution of compound 3 (84 mg, 0.22 mmol) and tridecyloxirane (302 mg, 1.34 mmol) in EtOH (2 mL) was added triethylamine (0.12 ml, 0.88 mmol). The reaction mixture was then irradiated in the microwave reactor at 150° C. for 5 h (FIG. 19B). Purification of the crude residue via flash column chromatography (gradient eluent: 1-2.0% of MeOH/DCM then 2.0-4.0% MeOH/DCM containing 0.5% NH₄OH) afforded cKK-E15 (200 mg, 78%) as a light-yellow oil. ¹H NMR H NMR (500 MHz, CDCl₃) δ 4.02-3.99 (m, 2H), 3.63-3.6 (m, 4H), 2.58-2.22 (m, 12H), 1.99-1.68 (m, 4H), 1.43-1.24 (m, 104H), 0.86 (t, J=6.9 Hz, 12H) (FIG. 19C). ¹³C NMR (125 MHz, CDCl3) δ 169.03, 168.74, 69.98, 69.62, 67.87, 67.64, 63.35, 63.06, 61.24, 60.93, 55.82, 54.72, 35.30, 35.06, 31.94, 29.92, 29.86, 29.74, 29.71, 29.69, 29.39, 25.86, 25.84, 25.76, 25.72, 22.70, 14.13; HRMS (ESI, m/z) calculated [M+H]+ for C72H145N406 1162.1159, found 1162.1153 (FIG. 19D).

avhh mRNA Synthesis.

mRNA was synthesized as previously described³⁴. Briefly, the GPI-anchored VHH sequence was ordered as a DNA gBlock from IDT (Integrated DNA Technologies) containing a 5′ UTR with Kozak sequence, a 3′ UTR derived from the mouse alpha-globin sequence, and extensions to allow for Gibson assembly. The sequence was human codon optimized using the IDT website. The gBlock was then cloned into a PCR amplified pMA7 vector through Gibson assembly using NEB Builder with 3 molar excess of insert. Gibson assembly reaction transcripts were 0.8% agarose gel purified prior to assembly reaction. Subsequent plasmids from each colony were Sanger sequenced to ensure sequence identity. Plasmids were digested into a linear template using NotI-HF (New England BioLabs) overnight at 37° C. Linearized templates were purified by ammonium acetate (Thermo Fisher Scientific) precipitation before being resuspended with nuclease-free water. In vitro transcription was performed overnight at 37° C. using the HiScribe T7 kit (NEB) following the manufacturer's instructions (full replacement of uracil with N1-methyl-pseudouridine). RNA product was treated with DNase I (Aldevron) for 30 min to remove template and purified using lithium chloride precipitation (Thermo Fisher Scientific). RNA transcripts were heat denatured at 65° C. for 10 min before being capped with a Cap1 structure using guanylyl transferase (Aldevron) and 2′-O-methyltransferase (Aldevron). Transcripts were then polyadenylated enzymatically (Aldevron). mRNA was then purified by lithium chloride precipitation, treated with alkaline phosphatase (NEB), and purified a final time. Concentrations were measured using a NanoDrop and mRNA stock concentrations were between 2 and 4 mg/mL. Purified RNA products were analyzed by gel electrophoresis to ensure purity. mRNA stocks were stored at −80° C.

### Nanoparticle Formulation.

Nanoparticles were formulated in a microfluidic device by mixing aVHH mRNA, DNA, the ionizable lipid, PEG, and cholesterol as previously described³³. Nanoparticles were made with variable mole ratios of these constituents. The nucleic acid (e.g., DNA barcode, mRNA) was diluted in 10 mM citrate buffer (Teknova) and loaded into a syringe (Hamilton Company). The materials making up the nanoparticles (CKK-E12, CKK-E15, cholesterol, 20a-hydroxycholesterol, C14PEG2K, C18PEG2K, DOPE) were diluted in ethanol and loaded into a second syringe. The citrate phase and ethanol phase were mixed in a microfluidic device using syringe pumps.

### DNA Barcoding.

Each chemically distinct LNP was formulated to carry its own distinct DNA barcode. For example, LNP-1 carried aVHH mRNA and DNA barcode 1, whereas the chemically distinct LNP-2 carried aVHH mRNA and DNA barcode 2. The DNA barcodes were designed rationally with universal primer sites and a specific 8-nucleotide (nt) barcode sequence, similar to what was previously described⁵⁰. DNA barcodes were single stranded, 91 nucleotides long, and purchased from Integrated DNA Technologies. Briefly, the barcodes had the following characteristics and modifications: i) nucleotides on the 5′ and 3′ ends were modified with a phosphorothioate to reduce exonuclease degradation, ii) universal forward and reverse primer regions were included to ensure equal amplification of each sequence, iii) 7 random nucleotides were included to monitor PCR bias, iv) a droplet digital PCR (ddPCR) probe site was included for ddPCR compatibility, and v) each barcode had a unique 8-nt barcode. An 8-nt sequence can generate over 48 (65,536) distinct barcodes. Only the 8-nucleotide sequences designed to prevent sequence bleaching and reading errors on the Illumina MiniSeg™ sequencing machine were used.

### Nanoparticle Characterization.

LNP hydrodynamic diameter and polydispersity index were measured using dynamic light scattering (DLS). LNPs were diluted in sterile 1× PBS to a concentration of ˜0.06 μg/mL and analyzed. LNPs were included if they met three criteria: diameter>20 nm, diameter<200 nm, and autocorrelation function with only one inflection point. Particles that met these criteria were pooled and dialyzed in 1× phosphate buffered saline (PBS, Invitrogen), and sterile filtered with a 0.22 μm filter. The nanoparticle concentration was determined using NanoDrop (Thermo Scientific).

### Encapsulation Efficiency.

Using two replicates for each LNP, 50 μL of a 6 ng/μL LNP-encapsulated RNA solution was added to 50 μL of a solution of 1× TE (Thermo Fisher) or a solution containing a 1:50 dilution of Triton X-100 (Sigma Aldrich). After incubating at 37° C. for 10 mn, 100 μL of a solution of 1:100 of RiboGreen reagent (Thermo Fisher) was added to each well. Fluorescence and absorbance were measured at an excitation wavelength of 485 nm and an emission wavelength of 528 nm with a plate reader (BioTek Synergy H4 Hybrid).

### Animal Experiments.

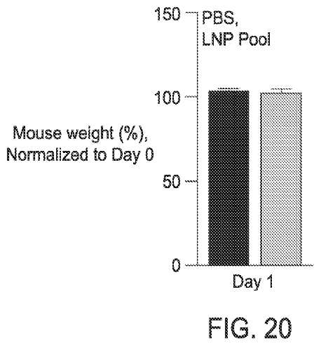

All animal experiments were performed in accordance with the Georgia Institute of Technology's Institutional Animal Care and Use Committee (IACUC). C57BL/6J (#000664) mice were purchased from the Jackson Laboratory. In all experiments, mice were aged 5-8 weeks, and N=4 mice per group were injected intravenously via the lateral tail vein. Weights for all mice for all experiments are included in FIG. 20.

### Cell Isolation.

In all cases, mice were sacrificed 1 day after administration of LNPs and immediately perfused with 20 mL of 1×PBS through the right atrium. The liver was isolated immediately following perfusion, minced with scissors, and then placed in a digestive enzyme solution with collagenase type I (Sigma Aldrich), collagenase XI (Sigma Aldrich), and hyaluronidase (Sigma Aldrich) at 37° C. and 750 rpm for 45 minutes. Digested tissues were passed through a 70 μm filter and red blood cells were lysed.

### Cell Staining.

Cells were stained to identify specific cell populations and sorted using a BD FacsFusion cell sorter. Antibody clones used for staining were anti-CD31 (390, BioLegend), anti-CD45.2 (104, BioLegend), anti-CD68 (FA-11, BioLegend), anti-aVHH (17A2, GenScript), live/dead (Thermo Fisher). Representative gating strategies for liver cell populations are included in FIG. 8A-B. To allow for pooling of samples into a single device, cells were stained with a streptavidin-conjugated H-2 MHC class I (M1/42, BioLegend) antibody, and biotinylated cell hash oligos were added at 0.5 μM final concentration after a single wash to remove unbound antibody.

### PCR Amplification for Traditional Barcoded LNP Analysis.

All samples were amplified and prepared for sequencing using a nested PCR protocol as previously described 51. More specifically, 1 μL of each primer (10 M reverse/forward) were added to 5 μL of Kapa HiFi 2× master mix, 2 μL sterile H2O, and 1 μL DNA template. The second PCR added Nextera XT chemistry, indices, and i5/i7 adapter regions and used the product from PCR 1 as template.

### Deep Sequencing.

Illumina deep sequencing was performed on Illumina MiniSeg™ using standard protocols suggested by Illumina. The sequencing was conducted in the Georgia Tech Molecular Evolution core.

### Nanoparticle Data Analysis & Statistics.

Sequencing results were processed using a custom Python-based tool to extract raw barcode counts for each tissue. These raw counts were then normalized with an R script prior to further analysis. Counts for each particle were normalized to the barcoded LNP mixture injected into mice, as previously described⁹. Statistical analyses were done using GraphPad Prism 7. Data is plotted as mean±standard error mean unless otherwise stated.

### Synthesis of Microwell-Seq Barcoded Beads.

To generate orthogonal beads containing 10% barcode binding sequences, the following protocol was used. Two milliliters of 50 M amine modified oligo (table 1) was conjugated to 150 mg of 20 μm carboxyl coated magnetic beads (kbspheretech) using 200 mg of EDC and NHS-ester (Sigma Aldrich) in 6 ml of 0.1M MES overnight. The conjugated beads were then washed once in 0.1M PBS containing 0.02% Tween-20 and two more times in TE (pH 8.0) using a magnet.

To add the three unique bead barcodes the conjugated beads were subjected to 3 rounds of split-pool PCR using the cell barcode oligos with the following protocol. The beads were washed once in ddH₂O and resuspended in 4.5 mL of 1× Kappa HF master mix, and 45 μL were aliquoted into a 96 well plate. Five microliters of 50 μM of a unique cell barcode oligo, with a complementary sequence to the amine modified oligo, was added, and amplified using the following PCR program: 94° C. for 5 min, 5 cycles of 94° C. for 15s, 50° C. for 4 min and 72° C. for 4 min and a final 4° C. hold. The beads were then pooled and washed twice with ddH₂O and repeated twice more with the additional plates of cell barcodes. The final set of cell barcodes also contained a unique molecular identifier (UMI) as well as a 15-nucleotide poly-T region for mRNA binding (FIG. 6B). To add the LNP barcode binding site, PCR was performed using the above method; however, a mixture of Poly-A oligo and Poly-A/LNP oligo was used for priming at a molar ratio of 10:1. After the final round of PCR the beads were pooled, washed twice in ddH₂O, and denatured in denaturation solution composed of 150 mM sodium hydroxide solution with 0.01% Tween 20 for 10 minutes at room temperature with rotation. The beads were then washed two times in denaturation solution followed by three washes with neutralization which contained 100 mM Tris (pH 8.0), 1 mM EDTA, and 0.010% Tween 20. The final beads were stored in 1×TE with 0.01% Tween 20 at 4° C. for up to one year.

### Device Generation and Bead Processing.

The generation of the microwell device and subsequent library preparation was performed following the protocol from Han et al. with a few modifications to accommodate CITE-seq and LNP barcode. The microwell device was generated using a PDMS 1 million-well device (iBioChips) to create a positive imprint mold for generation of a 5% agarose in PBS disposable device. One hundred thousand of the isolated and pooled cells were loaded onto the agarose device and allowed to settle for 10 minutes until most of the cells had fallen into the bottoms of the wells. Two washes were performed with ice-cold PBS to remove any cells that did not fall into a single well. The device was then placed on a strong magnet, and 1 million barcoded beads were slowly distributed over the device and allowed to incubate for 10 minutes so that most of the beads were immobilized into each well. Two more washes were performed to remove any unbound beads, and 1 mL of cold lysis buffer (0.1M Tris-HCL pH7.5, 0.5 M LiCl, 1% SDS, 10 mM EDTA and 5 mM dithiothreitol) was added and allowed to incubate on ice for 10 minutes. After lysis the device was cut out and flipped over, and the magnet was used to remove the beads from the wells. The beads were pooled, washed twice with 6×SSC, and given one final wash in 50 mM Tris-HCL pH 8.0.

### Library Preparation.

The pooled beads were then placed in a reverse transcription reaction containing 200 U M-MLV Reverse Transcriptase (BioChain Institute), 1× RT buffer, 20 U RNAse inhibitor (NEB), 1 M betaine (Sigma), 6 mM MgCl₂ (Sigma), 2.5 mM DTT (Thermo Fisher), 1 mM dNTP (NEB), and 1 μM TSO primer. The beads were incubated for 90 minutes at 42° C. followed by a hold at 4° C. with constant shaking at 500 RPM. After the reverse transcriptase step, enzyme was removed using 1×TE with 0.5% SDS followed by a wash in 1×TE with 0.01% Tween 20 and finally a wash in 100 mM Tris-HCl pH 8.0.

To remove any unused single-stranded oligo from the beads, they were treated with 200 U of exonuclease I (NEB) in 1× ExoI buffer for 60 minutes at 37° C. with 500 RPM shaking. Following the digestion, excess ExoI was removed using the previously described TE-SDS, TE-Tween 20 and Tris-HCL pH 8.0 washes. After removal of ExoI, the beads were resuspended in 200 μL of Platinum II hot-start master mix (Thermo Fisher) with IS-PCR, p7 Multi Barcode Rvs and Hash p7 Rvs primers, and the first-round PCR was performed using the following cycling conditions: one cycle at 94° C. for 2 minutes, 12 cycles of 94° C. for 15 seconds, 60° C. for 15 seconds, and 68° C. for 2 minutes. The sample was pooled, the beads were removed and discarded, and the sample was purified using 0.6× SPRI beads. The long RNA fragments were collected on the SPRI beads, while the shorter barcode and hash reads remained in the PCR supernatant; these were purified using 2.0× SPRI beads and saved for use during the final round PCR. The RNA sample was then treated with TN5 transposase to fragment and add on sequencing handles for subsequent PCR. Both the DNA and fragmented RNA sample were then amplified using a second round of PCR with non-hot-start Q5 high-fidelity polymerase (NEB), P7 Nextera index adapters, and Microwell P5 primer using the following cycling conditions: one cycle at 70° C. for 5 minutes, 12 cycles of 98° C. for 30 seconds, 58° C. for 30 seconds, and 72° C. for 90 seconds, with a final extension at 72° C. for 2 minutes. The samples were then purified using 0.8× SPRI beads, pooled at a 10:1 molar ratio of RNA to DNA, and finally sequenced on an Illumina HiSeq paired-end 150-cycle run.

### Processing of Single Cell Data & Statistics.

The data were processed using zUMIs (v 2.9.7) for the RNA mapping and counting and Salmon Alevin (v1.5.2) for the DNA barcode and cell hashes^{52, 53}. All samples were mapped to GRCm39, and only Exonic regions were counted. All output files were loaded into Seurat (v 4.0.4), and in summary, cells were log normalized to a scale factor of 10,000, then scaled using a linear transformation⁵⁴. This was followed by PCA dimensional reduction and t-SNE clustering and then exported using rBCS for further analysis in BBrowser2 (v2.9.23). Once in BBrowser2, the cell search tool was used to identify the cell types within each cluster, and gene expression profiles were compared within cell types of interest. Barcode counts were combined with RNA counts in Seurat and treated in a similar manner to other multimodal datasets such as CITE-seq.

### DISCUSSION

After synthesizing new lipids for LNPs, scientists typically formulate them into nanoparticles and test their ability to deliver drugs in vitro or in vivo. However, both FDA-approved, systemically administered siRNA therapies^{3, 4} that use delivery vehicles have required scientists to understand the genes that enable and enhance drug delivery. These findings, coupled with recent data demonstrating that LNP delivery substantially increases²⁴ or decreases^{25, 26} depending on cell state, strongly suggest that further insights into the biology of delivery are needed to improve clinical nanoparticles.

The testing shown in this example establishes a sequencing-based multiomic system capable of performing high-throughput in vivo nanoparticle delivery assays and analyzing the cellular response to nanoparticles, all with single-cell resolution. By marrying empirical drug delivery datasets to biological readouts, SENT-seq generated several lines of evidence that cell heterogeneity influences LNP-mediated mRNA delivery. These lines of evidence were enabled by one key advantage to SENT-seq: cells are defined by their transcriptional state instead of cell surface markers.

In this case, delivery to 17 cell subtypes in the liver was quantified; it is believed that such delivery has not been previously measured in these subtypes. But the same advantage can also serve to quantify delivery to, and therefore target, (i) rare cells including hematopoietic stem cells, basal cells, and circulating tumor cells, or (ii) cells defined by a particular, even complicated, transcriptional state, such as exhausted T cells⁴⁶. A second, related advantage is that SENT-seq may be helpful in quantifying delivery in larger animals that do not have established flow antibodies for cells of interest. This is distinct from previous assays, which rely on tissue-level delivery readouts or require FACS antibody panels to isolate cells of interest; these antibody panels are far less common for non-human primates (NHPs) and other large animals.

It is anticipated that SENT-seq may help elucidate the genes driving non-liver targeting to the lung^{10, 12}, spleen^{10, 13}, and bone marrow³² using LNP-based delivery vehicles. Although additional work needs to be completed, this ability to simultaneously read out high throughput nanoparticle delivery and the cellular response to nanoparticles may lead to new datasets and insights that improve mRNA therapeutics.

The present disclosure also pertains to and includes at least the following aspects:

Aspect 1. An in vivo method of identifying a lipid nanoparticle optimized based on cellular state, delivery profile, or both cellular state and delivery profile, for delivery into a specific single cell comprising:
(a) formulating a lipid nanoparticle, wherein the lipid nanoparticle comprises an identifying DNA barcode and a VHH antibody;
(b) administering a plurality of the lipid nanoparticles to cells in a non-human mammal;
(c) determining the delivery profile of the lipid nanoparticle at a single cell level using steps comprising:
   contacting the cells with an agent that detects the DNA barcode, the VHH antibody, and endogenous mRNA of the cell to identify one or more viable cells having the DNA barcode and the VHH antibody at a single cell level based on sequencing; and
   identifying the DNA barcode in the one or more viable cells to determine the composition of the lipid nanoparticle to correlate the composition of the lipid nanoparticle with the tissue or cell type containing the nanoparticle;
      and,
(d) determining the cellular state in one or more cells at a single cell level having been administered the lipid nanoparticles using steps comprising:
   measuring by sequencing expression of one or more of an inflammatory gene, a toxicity gene, and a cell state gene in the one or more viable cells having the DNA barcode and the VHH antibody; and
   identifying the lipid nanoparticle by correlating reduced expression of the one or more one of an inflammatory gene, a toxicity gene, and a cell state gene in a cell compared to a cell not administered the lipid nanoparticle with the composition of the nanoparticle, thereby identifying the lipid nanoparticle optimized based on cellular state and/or delivery profile for delivery into a specific single cell.
      Aspect 2. The method of aspect 1, further comprising measuring the expression of one or more of an inflammatory gene, a toxicity gene, and a cell state gene in a cell that has not been contacted in the lipid nanoparticles.
      Aspect 3. The method of aspects 1 or 2, wherein the method comprises measuring the expression of at least one inflammatory gene, at least one toxicity gene, and at least one cell state gene.
      Aspect 4. The method of any one of aspects 1 to 3, wherein the inflammatory gene is selected from the group consisting of Apoa2, CD163, Dnajb9, Traf3, and combinations thereof.
      Aspect 5. The method of any one of aspects 1 to 3, wherein the inflammatory gene is one or more gene shown in Table 1.
      Aspect 6. The method of any one of aspects 1 to 5, wherein the toxicity gene is selected from the group consisting of Gsk3b, Rpto, Dnm1, Casp3, and combinations thereof.
      Aspect 7. The method of any one of aspects 1 to 5, wherein the toxicity gene is one or more gene shown in Table 2.
      Aspect 8. The method of any one of aspects 1 to 7, wherein the cell state gene is selected from the group consisting of CDk9, Rdx, Ldir, Atm, and combinations thereof.
      Aspect 9. The method of any one of aspects 1 to 7, wherein the cell state gene is one or more gene shown in Table 3.
      Aspect 10. The method of any one of aspects 1 to 9 further comprising measuring expression of one or more gene indicative of endocytosis and measuring the expression of one or more of an inflammatory gene, a toxicity gene, and a cell state gene in the viable cells having the DNA barcode and the VHH antibody.
      Aspect 11. The method of aspect 10, wherein increased expression of one more gene indicative of endocytosis when compared to a cell not administered the lipid nanoparticle is indicative of a lipid nanoparticle having improved uptake in the cell.
      Aspect 12. The method of aspects 10 or 11, wherein the one of more gene indicative of endocytosis is one or more gene shown in Table 4.
      Aspect 13. The method of any one of aspects 1 to 12, wherein the method identifies lipid nanoparticles that do not induce toxicity or immune activation.
      Aspect 14. The method of any one of aspects 1 to 13, wherein the method comprises simultaneously identifying the DNA barcode in the cell and measuring expression of the one or more of an inflammatory gene, a toxicity gene, and a cell state gene in the viable cells having the DNA barcode and the VHH antibody.
      Aspect 15. The method of any one of aspects 1 to 15, wherein the agent that simultaneously detects the DNA barcode, the VHH antibody, and the endogenous mRNA of the cells is a bead having a capture sequence with a poly-T end (a PolyA binding site) and a capture sequence with a DNA barcode capture site.
      Aspect 16. The method of aspect 15, wherein the DNA barcode capture site is capable of binding a universal sequence found in all of the DNA barcodes.
      Aspect 17. The method of aspects 15 or 16, wherein the poly-T end detects the VHH antibody and endogenous mRNA of the cell.
      Aspect 18. The method of any one of aspects 15-17, wherein the DNA barcode capture site comprises or consists of SEQ ID NO: 1.
      Aspect 19. The method of any one of aspects 15-18, wherein the poly-T end comprises or consists of SEQ ID NO: 2
      Aspect 20. The method of aspect 15, wherein the bead is a carboxyl-coated magnetic polymer bead.
      Aspect 21. The method of any one of aspects 1-20, wherein the method does not comprise measuring protein levels.
      Aspect 22. The method of any one of aspects 1-21, further comprising quantifying the lipid nanoparticles in the single cell.
      Aspect 23. A bead for characterizing a lipid nanoparticle, having a capture sequence with a poly-T end (a PolyA binding site) and a capture sequence with a DNA barcode capture site linked to the bead.
      Aspect 24. The bead of aspect 23, wherein the DNA barcode capture site is capable of binding a universal sequence DNA sequence found in DNA barcodes.
      Aspect 25. The bead of aspects 23 or 24, wherein the bead is a carboxyl-coated magnetic polymer bead coated with an amine reactive oligo composed of three bead barcodes (BC1-3), a sequencing adapter (GT), two linker sequences (L1-2), an UMI and PolyA binding site and/or DNA barcode binding site, wherein the DNA barcode binding site comprises the nucleotide sequence of SEQ ID NO: 1.
      Aspect 26. The bead of aspect 25, wherein the bead comprises a Poly A binding site and a DNA barcode binding site.
      Aspect 27. The bead of any one of aspects 23-26, wherein the poly-T end detects a VHH antibody and endogenous mRNA of the cell.
      Aspect 28. A kit for characterizing a lipid nanoparticles for in vivo delivery of an agent comprising a bead of any one of aspects 23-27.

### REFERENCES AND NOTES

1. Baden, L. R. et al. Efficacy and Safety of the mRNA-1273 SARS-CoV-2 Vaccine. The New England Journal of Medicine (2020).
2. Polack, F. P. et al. Safety and Efficacy of the BNT162b2 mRNA Covid-19 Vaccine. The New England Journal of Medicine 383, 2603-2615 (2020).
3. Gillmore, J. D. et al. CRISPR-Cas9 In Vivo Gene Editing for Transthyretin Amyloidosis. The New England Journal of Medicine (2021).
4. Adams, D. et al. Patisiran, an RNAi Therapeutic, for Hereditary Transthyretin Amyloidosis. The New England Journal of Medicine 379, 11-21 (2018).
5. Translate Bio Announces Results from Second Interim Data Analysis from Ongoing Phase 1/2 Clinical Trial of MRT5005 in Patients with Cystic Fibrosis (CF). https://investors.translate.bio/news-releases/news-release-details/translate-bio-announces-results-second-interim-data-analysis.
6. Translate Bio Announces Pipeline Program Update. https://investors.translate.bio/news-releases/news-release-details/translate-bio-announces-pipeline-program-update.
7. Arcturus Therapeutics Announces First Quarter 2021 Company Overview and Financial Results and Provides New Clinical Data. https://ir.arcturusrx.com/news-releases/news-release-details/arcturus-therapeutics-announces-first-quarter-2021-company.
8. Altmoglu, S., Wang, M. & Xu, Q. Combinatorial library strategies for synthesis of cationic lipid-like nanoparticles and their potential medical applications. Nanomedicine (Lond) 10, 643-657 (2015).
9. Lokugamage, M. P., Sago, C. D. & Dahlman, J. E. Testing thousands of nanoparticles in vivo using DNA barcodes. Current Opinion in Biomedical Engineering 7, 1-8 (2018).
10. Cheng, Q. et al. Selective organ targeting (SORT) nanoparticles for tissue-specific mRNA delivery and CRISPR-Cas gene editing. Nature nanotechnology 15, 313-320 (2020).
11. Sago, C. D. et al. High-throughput in vivo screen of functional mRNA delivery identifies nanoparticles for endothelial cell gene editing. Proceedings of the National Academy of Sciences (2018).
12. Kauffman, K. J. et al. Rapid, Single-Cell Analysis and Discovery of Vectored mRNA Transfection In Vivo with a loxP-Flanked tdTomato Reporter Mouse. Molecular therapy. Nucleic acids 10, 55-63 (2018).
13. Kranz, L. M. et al. Systemic RNA delivery to dendritic cells exploits antiviral defence for cancer immunotherapy. Nature 534, 396-401 (2016).
14. Veiga, N. et al. Cell specific delivery of modified mRNA expressing therapeutic proteins to leukocytes. Nature communications 9, 4493 (2018).
15. Paunovska, K. et al. Nanoparticles Containing Oxidized Cholesterol Deliver mRNA to the Liver Microenvironment at Clinically Relevant Doses. Advanced materials, e1807748 (2019).
16. Akinc, A. et al. Targeted delivery of RNAi therapeutics with endogenous and exogenous ligand-based mechanisms. Molecular therapy: the journal of the American Society of Gene Therapy 18, 1357-1364 (2010).
17. Akinc, A. et al. The Onpattro story and the clinical translation of nanomedicines containing nucleic acid-based drugs. Nature nanotechnology 14, 1084-1087 (2019).
18. Nair, J. K. et al. Multivalent N-acetylgalactosamine-conjugated siRNA localizes in hepatocytes and elicits robust RNAi-mediated gene silencing. J Am Chem Soc 136, 16958-16961 (2014).
19. Balwani, M. et al. Phase 3 Trial of RNAi Therapeutic Givosiran for Acute Intermittent Porphyria. The New England Journal of Medicine 382, 2289-2301 (2020).
20. Garrelfs, S. F. et al. Lumasiran, an RNAi Therapeutic for Primary Hyperoxaluria Type 1. The New England Journal of Medicine 384, 1216-1226 (2021).
21. Ray, K. K. et al. Two Phase 3 Trials of Inclisiran in Patients with Elevated LDL Cholesterol. The New England Journal of Medicine 382, 1507-1519 (2020).
22. Alnylam Announces U.S. Food and Drug Administration Acceptance of New Drug Application for Investigational Vutrisiran for the Treatment of the Polyneuropathy of Hereditary ATTR Amyloidosis. https://investors.alnylam.com/press-release?id=25811.(2021).
23. Pasi, K. J. et al. Targeting of Antithrombin in Hemophilia A or B with RNAi Therapy. The New England Journal of Medicine 377, 819-828 (2017).
24. Patel, S. et al. Boosting Intracellular Delivery of Lipid Nanoparticle-Encapsulated mRNA. Nano letters 17, 5711-5718 (2017).
25. Paunovska, K. et al. Increased PIP3 activity blocks nanoparticle mRNA delivery. Science advances 6, eaba5672 (2020).
26. Lokugamage, M. P. et al. Mild Innate Immune Activation Overrides Efficient Nanoparticle-Mediated RNA Delivery. Advanced materials, e1904905 (2019).
27. Paunovska, K. et al. A Direct Comparison of in Vitro and in Vivo Nucleic Acid Delivery Mediated by Hundreds of Nanoparticles Reveals a Weak Correlation. Nano letters 18, 2148-2157 (2018).
28. Katzenelenbogen, Y. et al. Coupled scRNA-Seq and Intracellular Protein Activity Reveal an Immunosuppressive Role of TREM2 in Cancer. Cell 182, 872-885.e819 (2020).
29. Papalexi, E. & Satija, R. Single-cell RNA sequencing to explore immune cell heterogeneity. Nature reviews. Immunology 18, 35-45 (2018).
30. Cherry, C. et al. Computational reconstruction of the signalling networks surrounding implanted biomaterials from single-cell transcriptomics. Nature biomedical engineering (2021).
31. Paunovska, K. et al. Analyzing 2000 in Vivo Drug Delivery Data Points Reveals Cholesterol Structure Impacts Nanoparticle Delivery. ACS nano 12, 8341-8349 (2018).
32. Sago, C. D. et al. Nanoparticles that deliver RNA to bone marrow identified by in vivo directed evolution. J Am Chem Soc (2018).
33. Chen, D. et al. Rapid discovery of potent siRNA-containing lipid nanoparticles enabled by controlled microfluidic formulation. J Am Chem Soc 134, 6948-6951 (2012).
34. Tiwari, P. M. et al. Engineered mRNA-expressed antibodies prevent respiratory syncytial virus infection. Nature communications 9, 3999 (2018).
35. Sago, C. D. et al. Modifying a Commonly Expressed Endocytic Receptor Retargets Nanoparticles in Vivo. Nano letters (2018).
36. Stoeckius, M. et al. Simultaneous epitope and transcriptome measurement in single cells. Nature Methods 14, 865-868 (2017).
37. Zhang, Y., Sun, C., Wang, C., Jankovic, K. E. & Dong, Y. Lipids and Lipid Derivatives for RNA Delivery. Chemical reviews (2021).
38. Han, X. et al. Mapping the Mouse Cell Atlas by Microwell-Seq. Cell 173, 1307 (2018).
39. Kalucka, J. et al. Single-Cell Transcriptome Atlas of Murine Endothelial Cells. Cell 180, 764-779.e720 (2020).
40. Szklarczyk, D. et al. The STRING database in 2017: quality-controlled protein-protein association networks, made broadly accessible. Nucleic Acids Res 45, D362-d368 (2017).
41. Malumbres, M. Cyclin-dependent kinases. Genome biology 15, 122-122 (2014).
42. Fan, Z. et al. CDK13 cooperates with CDK12 to control global RNA polymerase II processivity. Science advances 6, eaaz5041 (2020).
43. Gilleron, J. et al. Image-based analysis of lipid nanoparticle-mediated siRNA delivery, intracellular trafficking and endosomal escape. Nat Biotechnol 31, 638-646 (2013).
44. Wittrup, A. et al. Visualizing lipid-foinulated siRNA release from endosomes and target gene knockdown. Nat Biotechnol 33, 870-876 (2015).
45. Hatit, M. Z. C. et al. Species-dependent in vivo mRNA delivery and cellular responses to nanoparticles. Accepted, Nature Nanotechnology (2021).
46. Blank, C. U. et al. Defining ‘T cell exhaustion’. Nature Reviews Immunology 19, 665-674 (2019).
47. Herrmann, I. K., Wood, M. J. A. & Fuhrmann, G. Extracellular vesicles as a next-generation drug delivery platform. Nature nanotechnology 16, 748-759 (2021).
48. Nooraei, S. et al. Virus-like particles: preparation, immunogenicity and their roles as nanovaccines and drug nanocarriers. Journal of Nanobiotechnology 19, 59 (2021).
49. Segel, M. et al. Mammalian retrovirus-like protein PEG10 packages its own mRNA and can be pseudotyped for mRNA delivery. Science 373, 882 (2021).
50. Dahlman, J. E. et al. Barcoded nanoparticles for high throughput in vivo discovery of targeted therapeutics. PNAS 114, 2060-2065 (2017).
51. Paunovska, K. et al. Nanoparticles Containing Oxidized Cholesterol Deliver mRNA to the Liver Microenvironment at Clinically Relevant Doses. Advanced Materials, 1807748 (2019).
52. Parekh, S., et al. A fast and flexible pipeline to process RNA sequencing data with UMIs. Gigascience 7 (2018).
53. Srivastava, A., et al. Alevin efficiently estimates accurate gene abundances from dscRNA-seq data. Genome biology 20, 65 (2019).
54. Hao, Y. et al. Integrated analysis of multimodal single-cell data. Cell 184, 3573-3587.e3529 (2021).

It is to be understood that while the disclosure has been described in conjunction with the preferred specific embodiments thereof, that the foregoing description and the examples that follow are intended to illustrate and not limit the scope of the disclosure. It will be understood by those skilled in the art that various changes may be made, and equivalents may be substituted without departing from the scope of the disclosure, and further that other aspects, advantages and modifications will be apparent to those skilled in the art to which the disclosure pertains. In addition to the embodiments described herein, the present disclosure contemplates and claims those inventions resulting from the combination of features of the disclosure cited herein and those of the cited prior art references which complement the features of the present disclosure. Similarly, it will be appreciated that any described material, feature, or article may be used in combination with any other material, feature, or article, and such combinations are considered within the scope of this disclosure.

The disclosures of each patent, patent application, and publication cited or described herein are hereby incorporated herein by reference, each in its entirely, for all purposes.

1. An in vivo method of identifying a lipid nanoparticle optimized based on cellular state, delivery profile, or both cellular state and delivery profile, for delivery into a specific single cell comprising:
(a) formulating a lipid nanoparticle, wherein the lipid nanoparticle comprises an identifying DNA barcode and a VHH antibody;
(b) administering a plurality of the lipid nanoparticles to cells in a non-human mammal;
(c) determining the delivery profile of the lipid nanoparticle at a single cell level using steps comprising:
contacting the cells with an agent that detects the DNA barcode, the VHH antibody, and endogenous mRNA of the cell to identify one or more viable cells having the DNA barcode and the VHH antibody at a single cell level based on sequencing; and
identifying the DNA barcode in the one or more viable cells to determine the composition of the lipid nanoparticle to correlate the composition of the lipid nanoparticle with the tissue or cell type containing the nanoparticle;
and,
(d) determining the cellular state in one or more cells at a single cell level having been administered the lipid nanoparticles using steps comprising:
measuring by sequencing expression of one or more of an inflammatory gene, a toxicity gene, and a cell state gene in the one or more viable cells having the DNA barcode and the VHH antibody; and
identifying the lipid nanoparticle by correlating reduced expression of the one or more one of an inflammatory gene, a toxicity gene, and a cell state gene in a cell compared to a cell not administered the lipid nanoparticle with the composition of the nanoparticle, thereby identifying the lipid nanoparticle optimized based on cellular state and/or delivery profile for delivery into a specific single cell.

2. The method of claim 1, further comprising measuring the expression of one or more of an inflammatory gene, a toxicity gene, and a cell state gene in a cell that has not been contacted in the lipid nanoparticles.

3. The method of claim 1, wherein the method comprises measuring the expression of at least one inflammatory gene, at least one toxicity gene, and at least one cell state gene.

4. The method of claim 1, wherein the inflammatory gene is selected from the group consisting of Apoa2, CD163, Dnajb9, Traf3, and combinations thereof.

5. (canceled)

6. The method of claim 1, wherein the toxicity gene is selected from the group consisting of Gsk3b, Rpto, Dnm1, Casp3, and combinations thereof.

7. (canceled)

8. The method of claim 1, wherein the cell state gene is selected from the group consisting of CDk9, Rdx, Ldir, Atm, and combinations thereof.

9. (canceled)

10. The method of claim 1 further comprising measuring expression of one or more gene indicative of endocytosis and measuring the expression of one or more of an inflammatory gene, a toxicity gene, and a cell state gene in the viable cells having the DNA barcode and the VHH antibody.

11. (canceled)

12. (canceled)

13. The method of claim 1, wherein the method identifies lipid nanoparticles that do not induce toxicity or immune activation.

14. The method of claim 1, wherein the method comprises simultaneously identifying the DNA barcode in the cell and measuring expression of the one or more of an inflammatory gene, a toxicity gene, and a cell state gene in the viable cells having the DNA barcode and the VHH antibody.

15. The method of claim 1, wherein the agent that simultaneously detects the DNA barcode, the VHH antibody, and the endogenous mRNA of the cells is a bead having a capture sequence with a poly-T end (a PolyA binding site) and a capture sequence with a DNA barcode capture site.

16. (canceled)

17. The method of claim 15, wherein the poly-T end detects the VHH antibody and endogenous mRNA of the cell.

18. The method of claim 15, wherein the DNA barcode capture site comprises or consists of SEQ ID NO: 1.

19. The method of claim 15, wherein the poly-T end comprises or consists of SEQ ID NO: 2

20. The method of claim 15, wherein the bead is a carboxyl-coated magnetic polymer bead.

21. The method of claim 1, wherein the method does not comprise measuring protein levels.

22. The method of claim 1, further comprising quantifying the lipid nanoparticles in the single cell.

23. A bead for characterizing a lipid nanoparticle, having a capture sequence with a poly-T end (a PolyA binding site) and a capture sequence with a DNA barcode capture site linked to the bead.

24. (canceled)

25. The bead of claim 23, wherein the bead is a carboxyl-coated magnetic polymer bead coated with an amine reactive oligo composed of three bead barcodes (BC1-3), a sequencing adapter (GT), two linker sequences (L1-2), an UMI and PolyA binding site and/or DNA barcode binding site, wherein the DNA barcode binding site comprises the nucleotide sequence of SEQ ID NO: 1.

26. The bead of claim 25, wherein the bead comprises a PolyA binding site and a DNA barcode binding site.

27. The bead of claim 23, wherein the poly-T end detects a VHH antibody and endogenous mRNA of the cell.

28. (canceled)


SEQUENCE LISTING (9 sequences)

SEQ ID NO: 1 - DNA, 22 bp, synthetic construct
  source 1..22: mol_type = other DNA
tacgagagtatgcctgagcagg

SEQ ID NO: 2 - DNA, 20 bp, synthetic construct
  source 1..20: mol_type = other DNA
tttttttttttttttttttt

SEQ ID NO: 3 - DNA, 121 bp, synthetic construct
  source 1..121: mol_type = other DNA
  misc_difference 48..53: note = b is c or g or t, not a
  misc_difference 69..74: note = b is c or g or t/u, not a
  misc_difference 90..95: note = b is c or g or t/u, not a
  misc_difference 96..101: note = b is c or g or t/u, not a
tttagggataacagggtaataagcagtggtatcaacgcagagtacgtbbbbbbcgactcactacagggbbbbbbtcggtgacacgatcgbbbbbbnnnnnntttttttttttttttttttt

SEQ ID NO: 4 - DNA, 86 bp, synthetic construct
  source 1..86: mol_type = other DNA
tttagggataacagggtaataagcagtggtatcaacgcagagtacccatagttgcgtctcatgcaaaatccgctgagtgatgtccc

SEQ ID NO: 5 - DNA, 104 bp, synthetic construct
  source 1..104: mol_type = other DNA
  misc_difference 84..89: note = b is c or g or t/u, not a
tttagggataacagggtaataagcagtggtatcaacgcagagtacgttttaggcgactcactacaggggctgagtgatgtcccbbbbbbagccactgtgctagc

SEQ ID NO: 6 - skipped

SEQ ID NO: 7 - DNA, 144 bp, synthetic construct
  source 1..144: mol_type = other DNA
  misc_difference 69..74: note = b is c or g or t/u, not a
  misc_difference 90..95: note = b is c or g or t/u, not a
  misc_difference 96..101: note = n is a or c or g or t
tttagggataacagggtaataagcagtggtatcaacgcagagtacgttttaggcgactcactacagggbbbbbbtcggtgacacgatcgbbbbbbnnnnnnttttttttttttttttttttctacgagagtatgcttgagcagg

SEQ ID NO: 8 - DNA, 160 bp, synthetic construct
  source 1..160: mol_type = other DNA
  misc_difference 48..53: note = b is c or g or t/u, not a
  misc_difference 90..95: note = b is c or g or t/u, not a
  misc_difference 96..101: note = n is a or c or g or t
  misc_difference 69..74: note = b is c or g or t/u, not a
tttagggataacagggtaataagcagtggtatcaacgcagagtacgtbbbbbbcgactcactacagggbbbbbbtcggtgacacgatcgbbbbbbnnnnnnttttttttttttttttttttaaaaaaaaaaaaaaaagatgctctcatacgaactcgtcc

SEQ ID NO: 9 - DNA, 97 bp, synthetic construct
  source 1..97: mol_type = other DNA
  misc_feature 1..6: note = phosphorothioate linkages (n) act to increase resistance to exonucleases
  misc_difference 27: note = n is a or c or g or t
  misc_difference 29: note = n is a or c or g or t
  misc_difference 57: note = n is a or c or g or t
  misc_difference 59: note = n is a or c or g or t
  misc_difference 69: note = n is a or c or g or t
  misc_difference 28: note = h is a or c or t, not g
  misc_difference 60: note = h is a or c or t, not g
  misc_difference 71: note = h is a or c or t, not g
  misc_difference 30: note = w is a or t
  misc_difference 58: note = w is a or t
  misc_difference 70: note = w is a or t
  misc_feature 91..96: note = phosphorothioate linkages (n) act to increase resistance to exonucleases
gnantngctctcatacgaactcgtccnhnwcctgctagtccacgtccatgtccaccnwnhtgatattgnwhgtggttagtcgagcagagacntnang